(19) United States
(12) Patent Application Publication
(10) Pub. No.: US 2025/0223327 A1
(43) Pub. Date: Jul. 10, 2025

(54) MICROBIAL PRODUCTION OF TITIN FIBERS WITH EXCEPTIONAL MECHANICAL PERFORMANCE

(71) Applicant: Washington University, St. Louis, MO (US)

(72) Inventors: Fuzhong Zhang, St. Louis, MO (US); Christopher H. Bowen, St. Louis, MO (US); Cameron Sargent, St. Louis, MO (US)

(21) Appl. No.: 18/036,695

(22) PCT Filed: Nov. 15, 2021
(86) PCT No.: PCT/US2021/059427

§ 371 Date: May 12, 2023

Related U.S. Application Data
(60) Provisional application No. 63/113,267, filed on Nov. 13, 2020.

(51) Int. Cl. C07K 14/47; C12N 15/70; D01F 6/68

(52) U.S. Cl. CPC C07K 14/4716; C12N 15/70; D01F 6/68

(57) ABSTRACT

Disclosed herein is a synthetic biology approach enabling microbial production of titin proteins, including titin oligomers and titin polymers, that yield fibers with exceptional mechanical properties derived from a unique structure-function relationship.

### CROSS-REFERENCE TO RELATED APPLICATIONS

This application is a U.S. National Phase Application of International Patent Application No. PCT/US2021/059427, filed Nov. 15, 2021, which claims the benefit of priority to U.S. Provisional Patent Application No. 63/113,267 filed Nov. 13, 2020, each of which are hereby incorporated by reference in their entirety.

### GOVERNMENT SUPPORT CLAUSE

This invention was made with government support under N000141912126 awarded by the Office of Naval Research and NNX15AU45G awarded by the National Aeronautics and Space Administration. The government has certain rights in the invention.

### SEQUENCE LISTING

The instant application contains a Sequence Listing which has been submitted electronically in ASCII form and is hereby incorporated by reference in its entirety. Said ASCII copy, created on Sep. 21, 2023, is named 2023-09-21_txt_15060-1573_Sequence_Listing.txt and is 3,971 bytes in size.

### FIELD OF THE DISCLOSURE

The field of the disclosure relates generally to microbially-produced proteins and fibers spun from microbially-produced proteins. More specifically, the field of the disclosure relates to split-intein-mediated microbial production of ultra-high molecular weight titin proteins and their wet-spinning into fibers.

### BACKGROUND OF THE DISCLOSURE

Biology is a great source of inspiration for materials design, as nature is capable of producing many high-performance, biodegradable materials from renewable feedstock through low-energy, aqueous processes. Examples include the exceptionally tough insect silks, underwater adhesive mussel byssus, compression-resistant abalone nacre, and highly elastic insect resilin. In many cases, these natural materials outperform the best available petroleum-based alternatives. Unfortunately, many of these high-performance natural materials cannot be easily harvested from their native sources, and their natural biosynthetic processes are often impossible to harness for scalable production as they are produced in limited quantity by slow-growing organisms. Thus, engineered microbial production strategies are needed to facilitate the practical use and development of these high-performance, renewable materials. While engineered microbes have been used successfully for scalable production of a great range of small-molecule compounds, the direct microbial production of polymeric materials with high mechanical performance has remained very limited. Many high-performance natural materials are protein-based and derive their superior mechanical performance from hierarchical assemblies of ultra-high molecular weight (UHMW) proteins with highly repetitive amino acid sequences. These UHMW, repetitive proteins are exceedingly difficult to produce in microbes due to genetic instability, low translation efficiency, and metabolic burden. The muscle protein titin, for example, endows muscle tissue with a combination of passive strength, damping capacity, and rapid mechanical recovery derived from titin's UHMW (>3 MDa) and highly repetitive sequence comprising hundreds of folded immunoglobulin (Ig) domains (FIG. 1A). While these appealing mechanical properties have inspired many efforts to engineer titin-like materials, titin's massive size and repetitive sequence have largely restricted these efforts to the production of titin-mimetic organic polymers rather than more environmentally-friendly protein-based materials (PBMs). In fact, previous recombinant production of titin proteins has only succeeded in expressing relatively short fragments (usually 8-12 Ig domains), and no prior efforts have been made to produce a macroscale material from microbially produced titin proteins.

### BRIEF DESCRIPTION OF THE DISCLOSURE

In one aspect, the present disclosure is directed to a microbially-synthesized titin protein comprising a plurality of folded immunoglobulin-like (Ig-like) domains. In some embodiments, the protein is an oligomer protein having at least about 4 Ig-like domains to at least about 20 Ig-like domains. In some embodiments, the oligomer protein has a molecular weight of at least about 40 kDa to at least about 150 kDa. In some embodiments, the protein is a polymer protein comprising more than about 20 Ig-like domains. In some embodiments, the polymer protein has a mass average molecular weight of at least 2 MDa, at least 3 MDa, at least 4 MDa, or at least 5 MDa.

In another aspect, the present disclosure is directed to a titin fiber comprising microbially-synthesized titin protein, wherein the microbially-synthesized titin protein comprises a plurality of folded immunoglobulin-like (Ig-like) domains. In some embodiments, the protein is an oligomer protein having at least about 4 Ig-like domains to at least about 20 Ig-like domains. In some embodiments, the oligomer protein has a molecular weight of at least about 40 kDa to at least about 150 kDa. In some embodiments, the titin fiber has a toughness of from about 15 MJ/m³ to about 75 MJ/m³, or from about 20 MJ/m³ to about 70 MJ/m³. In some embodiments, the titin fiber has an ultimate tensile strength of from 100 MPa to about 250 MPa, or from about 150 MPa to about 225 MPa. In some embodiments, the protein is a polymer protein comprising more than about 20 Ig-like domains. In some embodiments, the polymer protein has a mass average molecular weight of at least 2 MDa, at least 3 MDa, at least 4 MDa, or at least 5 MDa. In some embodiments, the titin fiber has a toughness of from about 115 MJ/m³ to about 150 MJ/m³, or from about 120 MJ/m³ to about 140 MJ/m³. In some embodiments, the titin fiber has an ultimate tensile strength of from about 350 MPa to about 500 MPa.

In yet another aspect, the present disclosure is directed to a process for synthesizing fibers from at least one microbially-produced titin protein, the process comprising dissolving the at least one microbially-produced titin protein in a denaturing solvent to produce a protein dope solution, wherein the at least one microbially-produced titin protein has a mass average molecular weight of at least about 40 kDa to at least about 5 MDa. In some embodiments, the at least one microbially-produced titin protein is selected from an oligomer protein having at least about 4 Ig-like domains to at least about 20 Ig-like domains having a molecular weight of at least about 40 kDa to at least about 150 kDa, a polymer protein comprising more than about 20 Ig-like domains having a mass average molecular weight of at least 2 MDa to about at least 5 MDa, and combinations thereof. In some embodiments, the denaturing solvent is hexafluoroisopropanol. In some embodiments, the process further comprises continuously spinning the microbially-synthesized titin polymer into fibers by extruding the polymer dope solution through a narrow-bore needle into a solvent. In some embodiments, the solvent is an aqueous solution. In some embodiments, the aqueous solution is water. In some embodiments, the process further comprises subjecting the fibers to a post-spin draw.

In an additional aspect, the present disclosure is directed to a method of synthesizing titin polymers, the method comprising: flanking an oligomer protein with a split-intein pair, wherein the oligomer protein is a titin subunit protein sequence comprising spatially-separated termini, and wherein the split-intein pair comprises a C-terminal half and an N-terminal half; overexpressing the flanked oligomer protein coding sequence in a protein expression system host cell culture to produce a chimeric Int^{C}-(oligomer protein)-Int^{N} protein; and polymerizing the flanked oligomer protein in vivo by a plurality of successive rounds of split-intein-catalyzed intermolecular ligation to produce titin polymers having a mass average molecular weight of at least 2 MDa. In some embodiments, the host cell is a protein-expressing microbial cell. In some embodiments, the protein-expressing microbial cell comprises an Escherichia coli cell, a Bacillus subtilis cell, a Saccharomyces cerevisiae cell, a Pichia pastoris cell. In some embodiments, the synthesized titin polymers comprise a plurality of folded Ig-like domains. In some embodiments, the synthesized titin polymers have a mass average molecular weight of at least 2 MDa, at least 3 MDa, at least 4 MDa, or at least 5 MDa. In some embodiments, the synthesized titin polymers have a toughness of from about 115 MJ/m³ to about 150 MJ/m³, or from about 120 MJ/m³ to about 140 MJ/m³. In some embodiments, the synthesized titin polymers have an ultimate tensile strength of from 350 MPa to about 500 MPa.

In yet another additional aspect, the present disclosure is directed to a method of synthesizing fibers from microbially-expressed titin oligomers, the method comprising: optimizing the coding sequence of titin oligomers; overexpressing the codon-optimized titin oligomers in a microbial host; and spinning purified titin oligomers into fibers. In some embodiments, the purified titin oligomers have a molecular weight between 40 kDa to 2 MDa.

### BRIEF DESCRIPTION OF THE DRAWINGS

The drawings described below illustrate various aspects of the disclosure.

FIG. 1A is an exemplary embodiment of the multi-scale structure of animal muscles in accordance with the present disclosure. Muscle tissue (1) is composed of specialized, elongated (>1 cm) cells called muscle fibers (2). Muscle fibers are packed with proteinaceous myofibrils (3) that span the entire length of the cell. Myofibrils are composed of repeating stacks of chemically controllable, contractile elements called sarcomeres (4). Sarcomeres are composed primarily of three proteins: actin, myosin, and titin. Titin spans half the length of the sarcomere, anchoring the opposing Z- and M-lines, and consists of hundreds of repeating immunoglobulin (Ig) domains that are integral to the passive strength (i.e., resistance to deformation without energy input), damping capacity, and mechanical recovery of the macroscopic muscle fiber.

FIG. 1B is an exemplary embodiment of a schematic representation of SI-based polymerization of the titin protein in E. coli in accordance with the present disclosure. To facilitate production of UHMW titin polymer in vivo, a relatively small, genetically stable, 41.3 kDa rabbit soleus titin protein coding sequence (41 g, SEQ ID NO. 1) was flanked by complimentary SIs, gp41-1^{C} (SEQ ID NO. 2) and gp41-1^{N} (SEQ ID NO. 3) (i). DNA sequence-recoded Int^{C}-4Ig-Int^{N} was produced in an engineered E. coli host under control of inducible promoter P_{LacO-1} (ii). The SI-flanked oligomer proteins were overexpressed in bioreactor cultures (iii) and polymerized intracellularly through successive rounds of SI-catalyzed intermolecular ligation to produce UHMW titin (iv). Purification and processing yielded titin fibers spun from microbially produced titin proteins that recapture the damping capacity and mechanical recovery of muscle along with exceptionally high strength and toughness (v).

FIG. 2 is an exemplary embodiment of dimensions from the titin 4Ig crystal structure in accordance with the present disclosure. Dimensions indicated are the Ig domain width (1.46 nm), inter-sheet distance (1.01 nm), inter-chain distance (0.46 nm), and 4Ig length (16.4 nm). Approximate dimensions were determined by analysis of the structure in UCSF Chimera.

FIG. 3A is an exemplary embodiment of a production and purification of titin oligomers and polymers including a 12% SDS-PAGE gel of un-induced and induced total cell lysates and HisTrap purified protein in accordance with the present disclosure.

FIG. 3B is an exemplary embodiment of a production and purification of titin oligomers and polymers including analytical SEC A280 chromatograms for oligomer, polymer, and known MW standards in accordance with the present disclosure.

FIG. 3C is an exemplary embodiment of a production and purification of titin oligomers and polymers including a SEC calibration curve of known MW standards herein in accordance with the present disclosure.

FIG. 4A is an exemplary embodiment of structural analyses of microbially produced UHMW titin protein and processed monofilament fibers including circular dichroism spectrum for purified titin polymer in water in accordance with the present disclosure. Inlaid pie graph indicates the results of spectral deconvolution by the BestSel program.

FIG. 4B is an exemplary embodiment of structural analyses of microbially produced UHMW titin protein and processed monofilament fibers including STEM image of purified titin polymer in accordance with the present disclosure. Scale bar is 50 nm.

FIG. 4C is an exemplary embodiment of structural analyses of microbially produced UHMW titin protein and processed monofilament fibers including SEM image of a fracture cross-section of the spun titin fiber in accordance with the present disclosure. Scale bar is 10 μm.

FIG. 4D is an exemplary embodiment of structural analyses of microbially produced UHMW titin protein and processed monofilament fibers including image of a textile net woven from the spun titin fibers in accordance with the present disclosure. White scale bar is 0.5 cm. Inset is a light microscopy image of an individual titin fiber. Black scale bar is 40 μm.

FIG. 4E is an exemplary embodiment of structural analyses of microbially produced UHMW titin protein and processed monofilament fibers including FTIR analysis of as-spun and post-spin drawn UHMW titin polymer fibers in accordance with the present disclosure. Averages of normalized spectra for each condition were overlaid.

FIG. 4F is an exemplary embodiment of structural analyses of microbially produced UHMW titin protein and processed monofilament fibers including deconvolved β-sheet content of titin polymer fibers in accordance with the present disclosure. For each fiber state, percentages were averaged for FTIR spectra acquired from three separate fibers. Error bars are the standard deviation of the three peak area calculations (see also FIGS. 8A and 8B).

FIG. 4G is an exemplary embodiment of structural analyses of microbially produced UHMW titin protein and processed monofilament fibers including Raman spectra of post-spin drawn titin polymer fibers oriented perpendicular (pink line) or parallel (blue line) to the polarization of the incident laser in accordance with the present disclosure. Spectra shown are the average of spectra acquired from three separate fibers. Standard deviations of the three measurements at each Raman shift are shown as black bars. The average ratio of the amide I peak (1670 cm⁻¹) intensity at 0° to that at 90° is shown above the spectrum as a measure of orientation sensitivity (see also FIGS. 9A and 9B).

FIG. 4H is an exemplary embodiment of structural analyses of microbially produced UHMW titin protein and processed monofilament fibers including synchrotron-based wide-angle X-ray diffraction analysis of spun titin polymer fibers, with a 1D radial intensity profile along the equator, with Gaussian fits for the (120) equatorial peak (dotted red), (200) equatorial peak (dotted blue), and two amorphous components (dotted gray) in accordance with the present disclosure. Inset shows the area selected for radial integration.

FIG. 4I is an exemplary embodiment of structural analyses of microbially produced UHMW titin protein and processed monofilament fibers including synchrotron-based wide-angle X-ray diffraction analysis of spun titin polymer fibers, with a 1D radial intensity profile along the meridian, with Gaussian fits for the (120) meridian peak (dotted red), (200) meridian peak (dotted blue), (002) peak (dotted pink), and two amorphous components (dotted gray) in accordance with the present disclosure. Inset shows the area selected for radial integration.

FIG. 4J is an exemplary embodiment of structural analyses of microbially produced UHMW titin protein and processed monofilament fibers including synchrotron-based wide-angle X-ray diffraction analysis of spun titin polymer fibers, with intensity as a function of azimuthal angle at the radial position of the equatorial (120) peak in accordance with the present disclosure. The peaks are fitted as sums of two Gaussians, corresponding to crystalline (narrow) and amorphous (broad) distributions. Small subsidiary peaks due to residual intensity from the (201) reflections (dotted purple) were treated as individual Gaussian functions. Inset shows the area selected for azimuthal integration.

FIG. 4K is an exemplary embodiment of structural analyses of microbially produced UHMW titin protein and processed monofilament fibers including synchrotron-based wide-angle X-ray diffraction analysis of spun titin polymer fibers, with intensity as a function of azimuthal angle at the radial position of the (200) peak in accordance with the present disclosure. The peaks are fitted as sums of two Gaussians, corresponding to crystalline (narrow) and amorphous (broad) distributions. Inset shows the area selected for azimuthal integration.

FIG. 5 is an exemplary embodiment of STEM analysis of purified UHMW titin polymer including dark-field negative stain STEM images of purified titin polymers in accordance with the present disclosure. Scale bars are 50 nm. Eluents from the HisTrap column were fully dialyzed against 5 mM ammonium bicarbonate and prepared for imaging as described herein.

FIG. 6 is an exemplary embodiment of distribution of UHMW titin polymer diameters measured by STEM in accordance with the present disclosure. Fibrils had an average cross-sectional diameter of 6.1 nm (+1.2 nm, n=376), which is consistent with fibril diameters found in previous studies (8-10 nm, see also FIG. 7).

FIG. 7 is an exemplary embodiment of SEM images of titin fibers spun from microbially produced UHMW titin proteins in accordance with the present disclosure. Fibers were imaged after pull tests as described herein. Scale bars are 10 μm. Fibers showed highly consistent size and morphology between samples. These images are representative of 12 fibers that were imaged using SEM.

FIG. 8A is an exemplary embodiment of FTIR analysis of titin fibers spun from microbially produced UHMW titin proteins using representative FTIR spectra from as-spun fibers in accordance with the present disclosure. Amide I bands were deconvolved into sets of 11 Lorentzian peaks.

FIG. 8B is an exemplary embodiment of FTIR analysis of titin fibers spun from microbially produced UHMW titin proteins using representative FTIR spectra from post-spin drawn fibers in accordance with the present disclosure. Amide I bands were deconvolved into sets of 11 Lorentzian peaks.

FIG. 9A is an exemplary embodiment of Raman spectromicroscopy analysis of as-spun titin fibers spun from microbially produced UHMW titin proteins in accordance with the present disclosure.

FIG. 9B is an exemplary embodiment of Raman spectromicroscopy analysis of post-spin drawn titin fibers spun from microbially produced UHMW titin proteins in accordance with the present disclosure. Raman spectra were acquired for fibers oriented perpendicular (pink line) or parallel (blue line) to the polarization of the incident laser. Spectra were normalized to the intensity of the peak at 1460 cm⁻¹, which corresponds to orientation insensitive CH2 bending. Spectra shown are the average of spectra acquired on three separate fibers for each fiber state. Standard deviations of the three measurements at each Raman shift are shown as black bars. As a measure of orientation sensitivity, the average ratio of the amide I peak (1670 cm⁻¹) intensity at 0° to that at 90° is shown above each spectrum. Black vertical lines represent the standard deviation of the three measures at each Raman shift.

FIG. 10A is an exemplary embodiment of titin fiber made from microbially produced UHMW titin protein WAXD data in accordance with the present disclosure. The 2D WAXD image for UHMW titin fiber reveals two broad but distinct equatorial reflections perpendicular to the fiber axis, along with a substantial amorphous component characteristic of a semi-crystalline material. Assuming an orthorhombic unit cell commonly applied to β-sheet crystallites in semi-crystalline fibers, the innermost equatorial peak is indexed as (200), corresponding to inter-sheet d-spacing along the unit cell a-axis, and the outermost equatorial peak is indexed as (120), corresponding to inter-chain d-spacing along the unit cell b-axis. The resulting center positions of the (200) and (120) crystalline peaks indicate a-axis inter-sheet d-spacing of 1.08 nm and b-axis inter-chain d-spacing of 0.46 nm, respectively. From the center position and FWHM of the (200) and (120) peaks, the Scherrer equation was used to determine the average crystallite size of 1.08 nm along the inter-sheet a-axis and 2.91 nm along the inter-chain b-axis, respectively (green lines indicate d-spacings, blue lines indicate calculated average crystallite sizes).

FIG. 10B is an exemplary embodiment of titin fiber made from microbially produced UHMW titin protein WAXD interpretation in accordance with the present disclosure. The resulting center positions of the (200) and (120) crystalline peaks indicate a-axis inter-sheet d-spacing of 1.08 nm and b-axis inter-chain d-spacing of 0.46 nm, respectively. From the center position and FWHM of the (200) and (120) peaks, the Scherrer equation was used to determine the average crystallite size of 1.08 nm along the inter-sheet a-axis and 2.91 nm along the inter-chain b-axis, respectively (green lines indicate d-spacings, blue lines indicate calculated average crystallite sizes).

FIG. 11A is an exemplary embodiment of mechanical testing of fibers spun from microbially produced 4Ig titin oligomer and UHMW titin polymer revealing exceptionally high toughness, damping capacity, and mechanical recovery reminiscent of natural muscle fibers including stress-strain curves from tensile tests of 14 fibers made from microbially produced UHMW titin protein (polymer) and 14 fibers made from low MW 4Ig titin (oligomer) in accordance with the present disclosure.

FIG. 11B is an exemplary embodiment of mechanical testing of fibers spun from microbially produced UHMW titin revealing exceptionally high toughness, damping capacity, and mechanical recovery reminiscent of natural muscle fibers including toughness measures extracted from the stress strain curves for polymer (purple) and 4Ig oligomer (gold) fibers in accordance with the present disclosure.

FIG. 11C is an exemplary embodiment of mechanical testing of fibers spun from microbially produced UHMW titin revealing exceptionally high toughness, damping capacity, and mechanical recovery reminiscent of natural muscle fibers including toughness of microbially produced (blue), natural (green), and man-made (gray) materials compared to that of the titin fibers made from UHMW titin proteins produced in the present disclosure (red).

FIG. 11D is an exemplary embodiment of mechanical testing of fibers spun from microbially produced UHMW titin revealing exceptionally high toughness, damping capacity, and mechanical recovery reminiscent of natural muscle fibers including loading/unloading curves for fibers made from microbially produced UHMW titin acquired at increasing strains from 0.6-30% in accordance with the present disclosure.

FIG. 11E is an exemplary embodiment of mechanical testing of fibers spun from microbially produced UHMW titin revealing exceptionally high toughness, damping capacity, and mechanical recovery reminiscent of natural muscle fibers including calculated damping capacity (blue curve) and damping energy (pink curve) at each strain tested in FIG. 11D, in accordance with the present disclosure. Error bars are the standard deviation of three loading/unloading cycles.

FIG. 11F is an exemplary embodiment of mechanical testing of fibers spun from microbially produced UHMW titin revealing exceptionally high toughness, damping capacity, and mechanical recovery reminiscent of natural muscle fibers including stress-strain curves for titin fibers made from microbially produced UHMW titin subjected to 11 consecutive loading/unloading cycles with one minute of humid (95% RH) air treatment between cycles in accordance with the present disclosure. Stress-strain curve of the first round is colored red. Following cycles use other colors.

FIG. 11G is an exemplary embodiment of mechanical testing of fibers spun from microbially produced UHMW titin revealing exceptionally high toughness, damping capacity, and mechanical recovery reminiscent of natural muscle fibers including calculated damping capacity (blue curve) and damping energy (pink curve) over consecutive cycles with humid air treatment between cycles in accordance with the present disclosure.

FIG. 12A is an exemplary embodiment of the ultimate tensile strength mechanical property of fibers spun from titin proteins (i.e., titin oligomers and UHMW titin polymers) in accordance with the present disclosure. Calculated from tensile tests of fibers made from 4Ig oligomers (gold), 8Ig oligomers (blue), 12Ig oligomers (green), and UHMW polymer (purple) proteins (n=14; horizontal lines denote, from top to bottom, upper fence, Q3, median, Q1, and lower fence; x denotes mean; other data indicated with circles). ***Unpaired two-tailed t-test P=4.4×10⁻¹⁵ for ultimate tensile strength.

FIG. 12B is an exemplary embodiment of the toughness mechanical property of fibers spun from titin proteins (i.e., titin oligomers and UHMW titin polymers) in accordance with the present disclosure. Calculated from tensile tests of fibers made from 4Ig oligomers (gold), 8Ig oligomers (blue), 12Ig oligomers (green), and UHMW polymer (purple) proteins (n=14; horizontal lines denote, from top to bottom, upper fence, Q3, median, Q1, and lower fence; x denotes mean; other data indicated with circles). ***Unpaired two-tailed t-test P=1.6×10⁻¹⁹ for toughness.

FIG. 12C is an exemplary embodiment of the elastic modulus mechanical property of fibers spun from titin proteins (i.e., titin oligomers and UHMW titin polymers) in accordance with the present disclosure. Calculated from tensile tests of fibers made from 4Ig oligomers (gold), 8Ig oligomers (blue), 12Ig oligomers (green), and UHMW polymer (purple) proteins (n=14; horizontal lines denote, from top to bottom, upper fence, Q3, median, Q1, and lower fence; x denotes mean; other data indicated with circles). ***Unpaired two-tailed t-test P=1.1×10⁻⁷ for modulus.

FIG. 12D is an exemplary embodiment of the breaking strain mechanical property of fibers spun from titin proteins (i.e., titin oligomers and UHMW titin polymers) in accordance with the present disclosure. Calculated from tensile tests of fibers made from 4Ig oligomers (gold), 8Ig oligomers (blue), 12Ig oligomers (green), and UHMW polymer (purple) proteins (n=14; horizontal lines denote, from top to bottom, upper fence, Q3, median, Q1, and lower fence; x denotes mean; other data indicated with circles). ***Unpaired two-tailed t-test P=2.1×10⁻¹² for breaking strain.

FIG. 13A is an exemplary embodiment of strength of the titin polymer fiber made from the microbially produced UHMW titin polymer compared to other man-made (grey), natural (green), and microbially produced (blue) materials (see also Table 8) in accordance with the present disclosure.

FIG. 13B is an exemplary embodiment of damping capacity of the titin polymer fiber made from the microbially produced UHMW titin polymer compared to other man-made (grey), natural (green), and microbially produced (blue) materials (see also Table 8) in accordance with the present disclosure.

FIG. 13C is an exemplary embodiment of damping capacity and mechanical recovery of fibers spun from titin oligomers, including loading/unloading curves for 4Ig titin (oligomer) fibers acquired at increasing strains from 1-16% in accordance with the present disclosure. Error bars are the standard deviation of the values measured at each cycle number for the three fiber samples that were tested.

FIG. 13D is an exemplary embodiment of damping capacity and mechanical recovery of fibers spun from titin oligomers, including average calculated damping capacity (blue curve) and damping energy (pink curve) at each strain tested in FIG. 13C. Error bars are the standard deviation of the three fiber samples tested at each strain in accordance with the present disclosure. Error bars are the standard deviation of the values measured at each cycle number for the three fiber samples that were tested.

FIG. 13E is an exemplary embodiment of damping capacity and mechanical recovery of fibers spun from titin oligomers, including stress-strain curves for 4Ig titin (oligomer) fibers subjected to 11 consecutive loading/unloading cycles with one minute of humid (95% RH) air treatment between cycles in accordance with the present disclosure. Stress-strain curve of the first round is colored red. Following cycles use other colors.

FIG. 13F is an exemplary embodiment of damping capacity and mechanical recovery of fibers spun from 4Ig titin oligomers, including average calculated damping capacity (blue curve) and damping energy (pink curve) of 4Ig oligomer titin fibers over consecutive cycles shown in FIG. 13E. in accordance with the present disclosure. Error bars are the standard deviation of the values measured at each cycle number for the three fiber samples that were tested.

FIG. 14A is an exemplary embodiment of a starting configuration of the molecular dynamics model of the titin fiber and its configuration after equilibration including a view from the x-axis of the all-atomistic model of titin fibrils (167-170) before equilibration starting from an anti-parallel configuration aligned along the y-axis in accordance with the present disclosure. The top two Ig domains of the red fibril are closest to the bottom two Ig domains of the blue fibril to create a staggered imbricated arrangement. Dimensions of the simulation box are 6.5 mm×16.6 mm×9.2 mm (x,y,z).

FIG. 14B is an exemplary embodiment of a starting configuration of the molecular dynamics model of the titin fiber and its configuration after equilibration including a view from the z-axis of the all-atomistic model of titin fibrils (167-170) before equilibration in accordance with the present disclosure. The left two fibrils are shifted replicates of the right two fibrils. Dimensions of the simulation box are 6.5×16.6×9.2 nm (x, y, z).

FIG. 14C is an exemplary embodiment of a starting configuration of the molecular dynamics model of the titin fiber and its configuration after equilibration including a view from the x-axis of model after equilibration in accordance with the present disclosure. Dimensions of the simulation boxes after equilibration are 4.0 mm×13.8 mm×4.6 mm (x,y,z).

FIG. 14D is an exemplary embodiment of a starting configuration of the molecular dynamics model of the titin fiber and its configuration after equilibration including a view from the z-axis of model after equilibration in accordance with the present disclosure. Dimensions of the simulation boxes after equilibration are 4.0×13.8× 4.6 nm (x, y, z).

FIG. 15A is an exemplary embodiment of molecular dynamics simulation of uniaxial tensile testing of a model titin fiber including representative uniaxial tensile stress-strain curves of the model titin fiber in accordance with the present disclosure.

FIG. 15B is an exemplary embodiment of molecular dynamics simulation of uniaxial tensile testing of a model titin fiber including snapshots of the molecular dynamics simulation of the titin fiber under tensile deformation in accordance with the present disclosure.

FIG. 15C is an exemplary embodiment of molecular dynamics simulation of uniaxial tensile testing of a model titin fiber including normalized atomic stress along the y-axis during extension of the titin fiber from 0% to 80% strain in accordance with the present disclosure. A selected Ig-like domain is shown in the red dashed box.

FIG. 15D is an exemplary embodiment of molecular dynamics simulation of uniaxial tensile testing of a model titin fiber including normalized atomic stress in along the y-axis during extension of the titin fiber from 0% to 80% strain in accordance with the present disclosure. A selected Ig-like domain is shown in the yellow dashed box.

FIG. 15E is an exemplary embodiment of molecular dynamics simulation of uniaxial tensile testing of a model titin fiber including changes in intra- and inter-fibril non-bonded energies (including Van der Waals, electrostatic, and hydrogen bonds) over the course of the simulation in accordance with the present disclosure. Error bars are the standard deviation of three trials.

FIG. 15F is an exemplary embodiment of molecular dynamics simulation of uniaxial tensile testing of a model titin fiber including total number of intra-fibril backbone-backbone hydrogen-bonds in the two selected Ig-like domains over the course of the tensile test. Error bars (shown as red or yellow bars) are the standard deviation of three trials in accordance with the present disclosure.

FIG. 16A is an exemplary embodiment of changes in non-bonded interactions and hydrogen bonds as a function of tensile strain in the titin fiber MD simulation including changes of intra-fibril (pink lines) and inter-fibril (blue lines) total non-bonded versus tensile strain in accordance with the present disclosure. Error bars are the standard deviation of three trials.

FIG. 16B is an exemplary embodiment of changes in non-bonded interactions and hydrogen bonds as a function of tensile strain in the titin fiber MD simulation including changes of intra-fibril (pink lines) and inter-fibril (blue lines) Van der Waals (Vdw) versus tensile strain in accordance with the present disclosure. Error bars are the standard deviation of three trials.

FIG. 16C is an exemplary embodiment of changes in non-bonded interactions and hydrogen bonds as a function of tensile strain in the titin fiber MD simulation including changes of intra-fibril (pink lines) and inter-fibril (blue lines) electrostatic energy differences versus tensile strain in accordance with the present disclosure. Error bars are the standard deviation of three trials.

FIG. 16D is an exemplary embodiment of changes in non-bonded interactions and hydrogen bonds as a function of tensile strain in the titin fiber MD simulation including changes of intra-fibril (red line) and inter-fibril (yellow line) hydrogen bond numbers between backbones (BB) in accordance with the present disclosure.

FIG. 16E is an exemplary embodiment of changes in non-bonded interactions and hydrogen bonds as a function of tensile strain in the titin fiber MD simulation including changes of intra-fibril (red line) and inter-fibril (yellow line) hydrogen bond numbers between backbones and sidechains (BS) in accordance with the present disclosure.

FIG. 16F is an exemplary embodiment of changes in non-bonded interactions and hydrogen bonds as a function of tensile strain in the titin fiber MD simulation including changes of intra-fibril (red line) and inter-fibril (yellow line) hydrogen bond numbers between sidechains (SS) in accordance with the present disclosure.

FIG. 16G is an exemplary embodiment of changes in non-bonded interactions and hydrogen bonds as a function of tensile strain in the titin fiber MD simulation including total changes in intra- and inter-fibril non-covalent bonding energies between 0 and 80% strain in accordance with the present disclosure.

FIG. 16H is an exemplary embodiment of changes in non-bonded interactions and hydrogen bonds as a function of tensile strain in the titin fiber MD simulation including total change in number of intra- or inter-fibril hydrogen bonds between 0 and 80% strain in accordance with the present disclosure. Hydrogen bonds formed between backbones (BB), between backbones and sidechains (BS) and between sidechains (SS).

FIG. 17 is an exemplary embodiment of a toughness upper limit calculation in accordance with the present disclosure.

FIG. 18A is an exemplary embodiment of equilibrated conformation calculated from GRAMM-X web server in accordance with the present disclosure.

FIG. 18B is an exemplary embodiment of aligned conformation from the disclosed model in accordance with the present disclosure.

FIG. 18C is an exemplary embodiment of a tensile stress-strain curve of face-to-face stacked titin fiber in accordance with the present disclosure.

FIG. 19A is an exemplary embodiment of a tensile force-displacement curve of a single titin fibril simulated by LAMMPS in accordance with the present disclosure.

FIG. 19B is an exemplary embodiment of a tensile force-displacement curve of a single titin fibril simulated by NAMD SMD in accordance with the present disclosure.

FIG. 19C is an exemplary embodiment of a schematic of the stretching of a single titin fiber in accordance with the present disclosure.

### DETAILED DESCRIPTION OF THE DISCLOSURE

Man-made high-performance polymers are typically non-biodegradable and derived from petroleum feedstock through energy intensive processes involving toxic solvents and byproducts. While engineered microbes have been used for renewable production of many small molecules, direct microbial synthesis of high-performance polymeric materials remains a major challenge. As disclosed herein, microbial production of megadalton muscle titin polymers has been engineered to yield high-performance fibers that not only recapture highly desirable properties of natural titin (i.e., high damping capacity and mechanical recovery) but also exhibit exceptionally high strength, toughness, and damping energy-outperforming many synthetic and natural polymers. Structural analyses and molecular modeling indicates these properties derive from unique inter-chain crystallization of folded immunoglobulin-like domains that resists inter-chain slippage while permitting intra-chain unfolding. These fibers have potential applications in areas from biomedicine to textiles, and the developed approach, coupled with the new structure-function insights, promises to accelerate further innovation in microbial production of high-performance materials.

In some embodiments of the present disclosure, a microbially-synthesized titin protein comprising a plurality of folded immunoglobulin-like (Ig-like) domains.

In some embodiments, the protein is an oligomer protein having at least about 4 Ig-like domains to at least about 20 Ig-like domains. In some embodiments, the oligomer protein has a molecular weight of at least about 40 kDa to at least about 150 kDa. In some embodiments, the protein is a polymer protein comprising more than about 20 Ig-like domains. In some embodiments, the polymer protein has a mass average molecular weight of at least 2 MDa, at least 3 MDa, at least 4 MDa, or at least 5 MDa.

In other embodiments of the present disclosure, a titin fiber comprising microbially-synthesized titin protein, wherein the microbially-synthesized titin protein comprises a plurality of folded immunoglobulin-like (Ig-like) domains.

In some embodiments, the protein is an oligomer protein having at least about 4 Ig-like domains to at least about 20 Ig-like domains. In some embodiments, the oligomer protein has a molecular weight of at least about 40 kDa to at least about 150 kDa. In some embodiments, the titin fiber has a toughness of from about 15 MJ/m³ to about 75 MJ/m³, or from about 20 MJ/m³ to about 70 MJ/m³. In some embodiments, the titin fiber has an ultimate tensile strength of from 100 MPa to about 250 MPa, or from about 150 MPa to about 225 MPa. In some embodiments, the protein is a polymer protein comprising more than about 20 Ig-like domains. In some embodiments, the polymer protein has a mass average molecular weight of at least 2 MDa, at least 3 MDa, at least 4 MDa, or at least 5 MDa. In some embodiments, the titin fiber has a toughness of from about 115 MJ/m³ to about 150 MJ/m³, or from about 120 MJ/m³ to about 140 MJ/m³. In some embodiments, the titin fiber has an ultimate tensile strength of from about 350 MPa to about 500 MPa.

In yet other embodiments of the present disclosure, a process for synthesizing fibers from at least one microbially-produced titin protein, the process comprising dissolving the at least one microbially-produced titin protein in a denaturing solvent to produce a protein dope solution, wherein the at least one microbially-produced titin protein has a mass average molecular weight of at least about 40 kDa to at least about 5 MDa.

In some embodiments, the at least one microbially-produced titin protein is selected from an oligomer protein having at least about 4 Ig-like domains to at least about 20 Ig-like domains having a molecular weight of at least about 40 kDa to at least about 150 kDa, a polymer protein comprising more than about 20 Ig-like domains having a mass average molecular weight of at least 2 MDa to about at least 5 MDa, and combinations thereof. In some embodiments, the denaturing solvent is hexafluoroisopropanol. In some embodiments, the process further comprises continuously spinning the microbially-synthesized titin polymer into fibers by extruding the polymer dope solution through a narrow-bore needle into a solvent. In some embodiments, the solvent is an aqueous solution. In some embodiments, the aqueous solution is water. In some embodiments, the process further comprises subjecting the fibers to a post-spin draw.

In additional embodiments of the present disclosure, a method of synthesizing titin polymers, the method comprising: flanking an oligomer protein with a split-intein pair, wherein the oligomer protein is a titin subunit protein sequence comprising spatially-separated termini, and wherein the split-intein pair comprises a C-terminal half and an N-terminal half; overexpressing the flanked oligomer protein coding sequence in a protein expression system host cell culture to produce a chimeric Int^{C}-(oligomer protein)-Int^{N} protein; and polymerizing the flanked oligomer protein in vivo by a plurality of successive rounds of split-intein-catalyzed intermolecular ligation to produce titin polymers having a mass average molecular weight of at least 2 MDa.

In some embodiments, the host cell is a protein-expressing microbial cell. In some embodiments, the protein-expressing microbial cell comprises an Escherichia coli cell, a Bacillus subtilis cell, a Saccharomyces cerevisiae cell, a Pichia pastoris cell. In some embodiments, the synthesized titin polymers comprise a plurality of folded Ig-like domains. In some embodiments, the synthesized titin polymers have a mass average molecular weight of at least 2 MDa, at least 3 MDa, at least 4 MDa, or at least 5 MDa. In some embodiments, the synthesized titin polymers have a toughness of from about 115 MJ/m³ to about 150 MJ/m³, or from about 120 MJ/m³ to about 140 MJ/m³. In some embodiments, the synthesized titin polymers have an ultimate tensile strength of from 350 MPa to about 500 MPa.

In yet other additional embodiments of the present disclosure, a method of synthesizing fibers from microbially-expressed titin oligomers, the method comprising: optimizing the coding sequence of titin oligomers; overexpressing the codon-optimized titin oligomers in a microbial host; and spinning purified titin oligomers into fibers.

In some embodiments, the purified titin oligomers have a molecular weight between 40 kDa to 2 MDa.

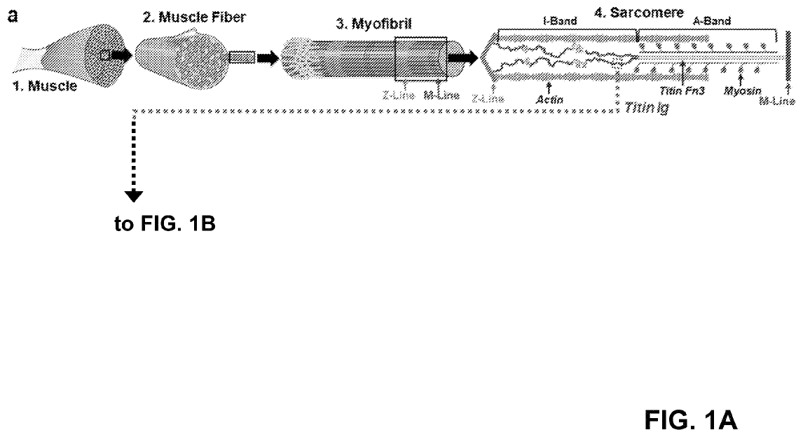

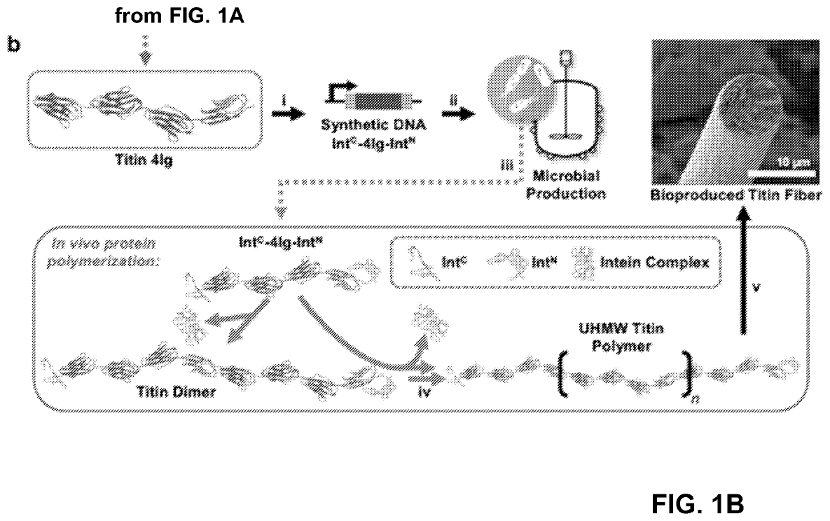

The challenge to potentiate microbial production of UHMW protein polymers is addressed for innovation of renewable high-performance materials throughout this disclosure. A synthetic biology approach is employed to mitigate the challenges of genetic instability and low translational efficiency through in vivo protein polymerization catalyzed by split-inteins (SI) in Escherichia coli (FIGS. 1A and 1B). In this manner, an engineered microbial production of UHMW titin proteins of unprecedented MW is achieved and subsequently an aqueous process is developed to spin the resulting polymers into high performance titin-based fibers that exhibit an intriguing combination of exceptional mechanical properties.

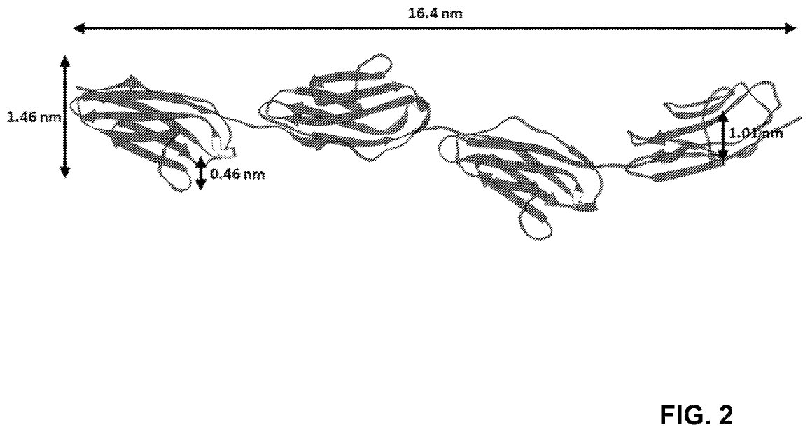

To enable efficient microbial production of UHMW titin proteins, the C- and N-terminal halves of a fast-reacting SI pair (gp41-1) were genetically fused to the N- and C-termini, respectively, of a relatively short titin subunit containing four Ig domains, yielding a chimeric protein Int^{C}-4Ig-Int^{N} (FIG. 1B). SIs catalyze spontaneous splicing reactions, covalently linking their fusion partners via a peptide bond and leaving only a few residues (≤6) at the ligation site with minimal effect on the properties of the resulting product. As disclosed herein, expressing this chimeric protein in E. coli, through multiple rounds of intracellular SI-catalyzed ligation of 4Ig subunits, produced UHMW titin proteins (FIG. 1B). To minimize intramolecular ligation and cyclization, a relatively rigid subunit of four Ig domains (167-170, hereafter 4Ig) from the I-band of the rabbit soleus muscle titin was polymerized (FIG. 1B). The crystal structure of 4Ig indicates structural rigidity and shows the N- and C-termini of the subunit spatially opposed and separated by approximately 16.4 nm (FIG. 2).

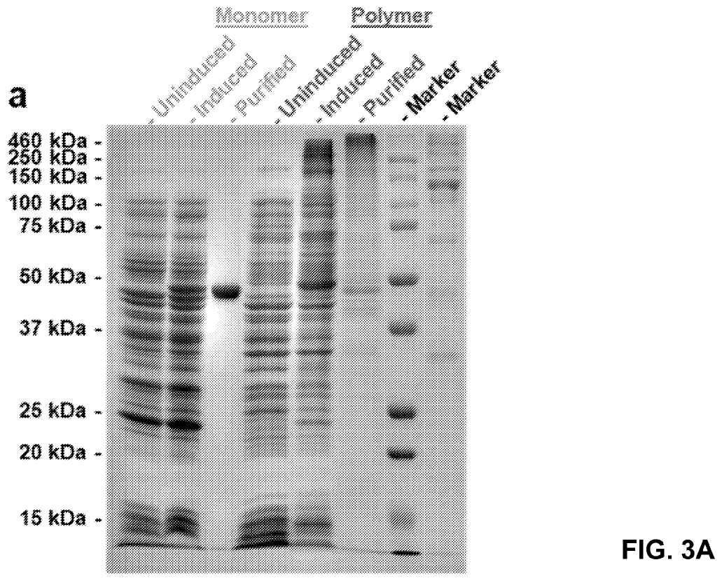

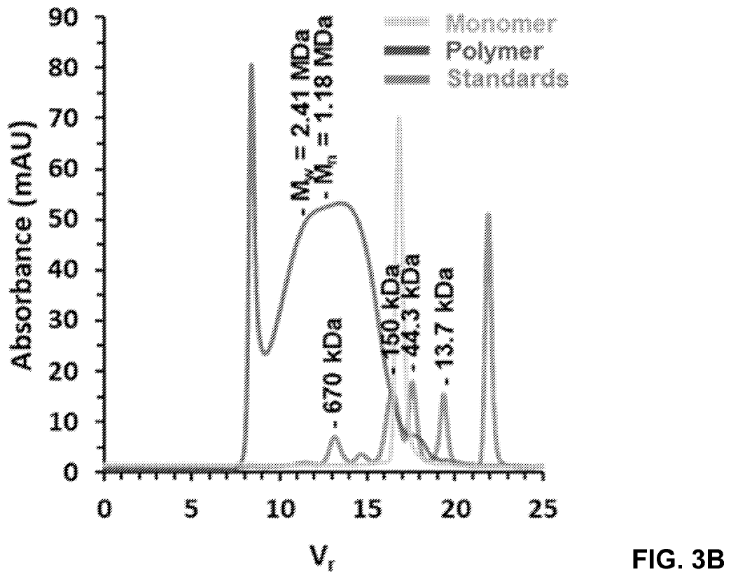

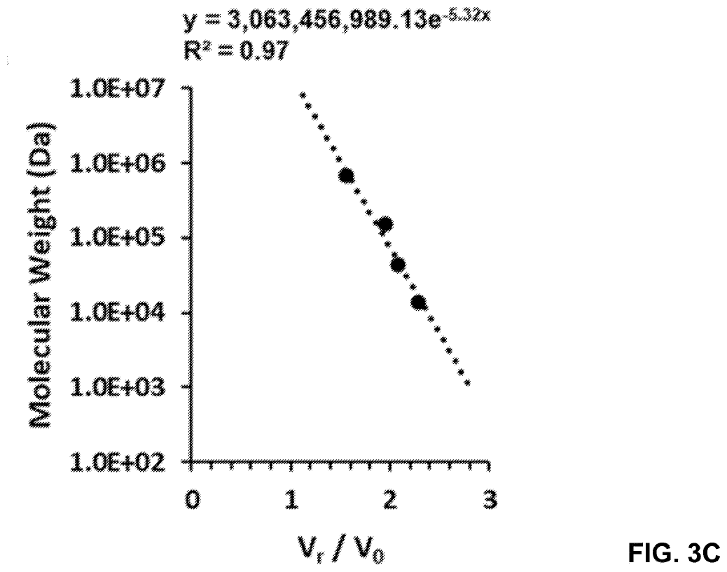

The chimeric protein Int^{C}-4Ig-Int^{N} and the 4Ig without SIs were expressed separately in E. coli shake flask cultures. SDS-PAGE analysis showed that the cells expressing the 4Ig oligomers produced a single band at the expected molecular weight of 43 kDa, while the Int^{C}-4Ig-Int^{N} expressing cells yielded a cluster of UHMW products up to and above 460 kDa. (FIG. 3A). Analytical size exclusion chromatography (SEC) confirmed that the purified titin polymers are indeed UHMW, with approximately 20% of the eluted species exceeding the column fractionation limit of 5 MDa (FIG. 3B). Even without considering this UHMW fraction, the mass average MW (M_{w}) of the titin polymer was estimated to be 2.4 MDa. Meanwhile, SEC analysis of the purified oligomers under identical conditions revealed a sharp eluent peak that corresponded roughly to the expected MW for the oligomers (43 kDa).

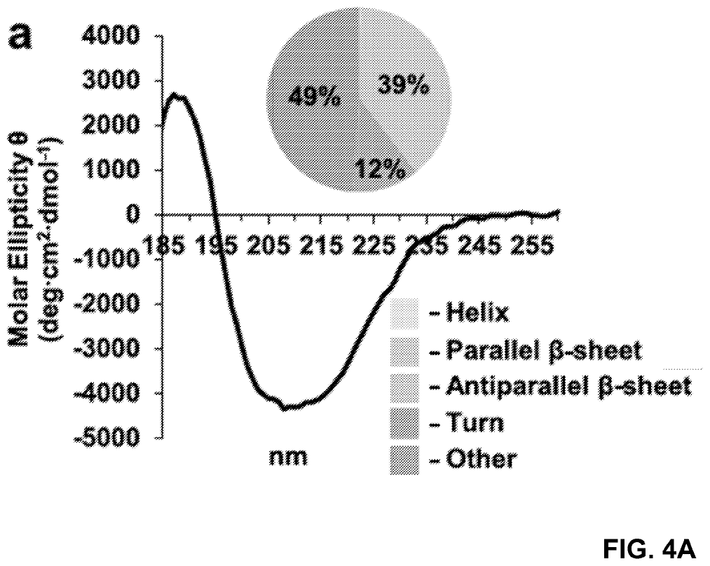

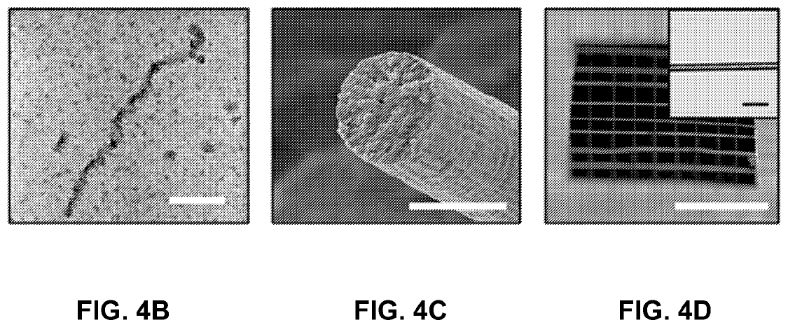

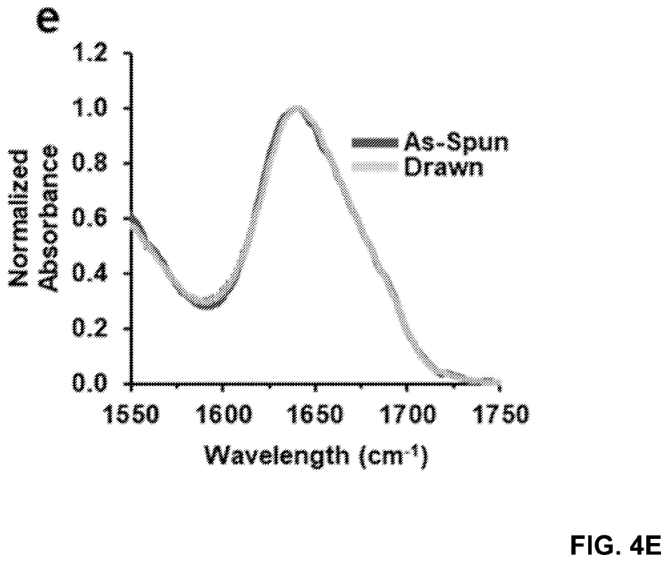

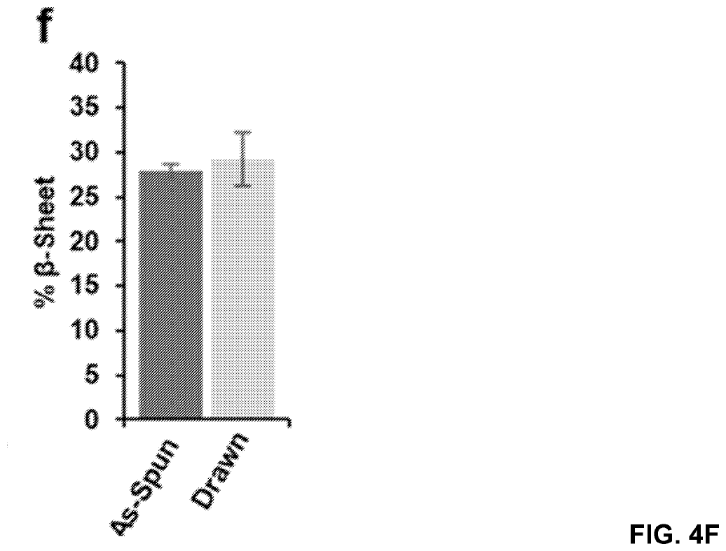

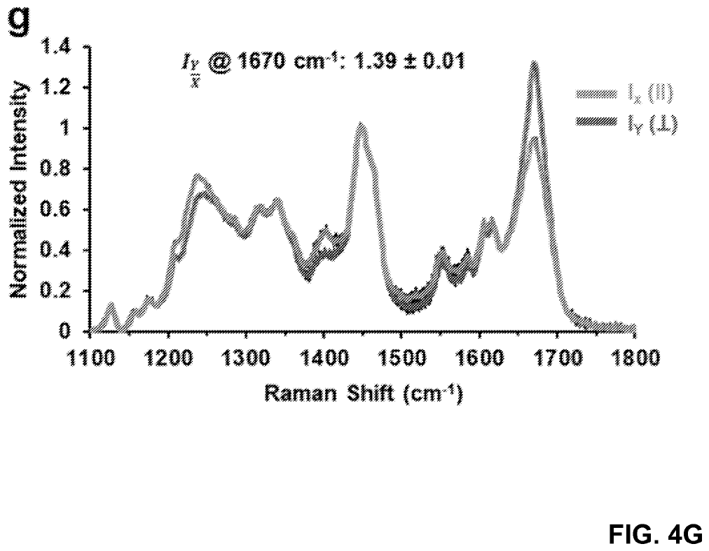

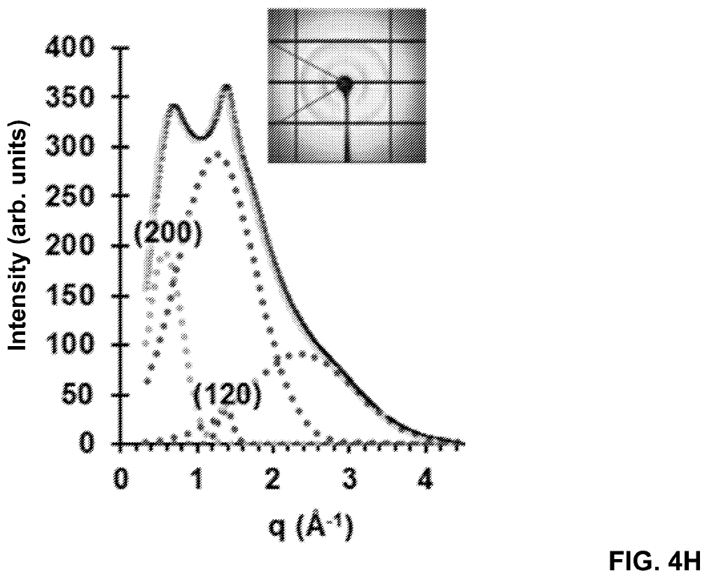

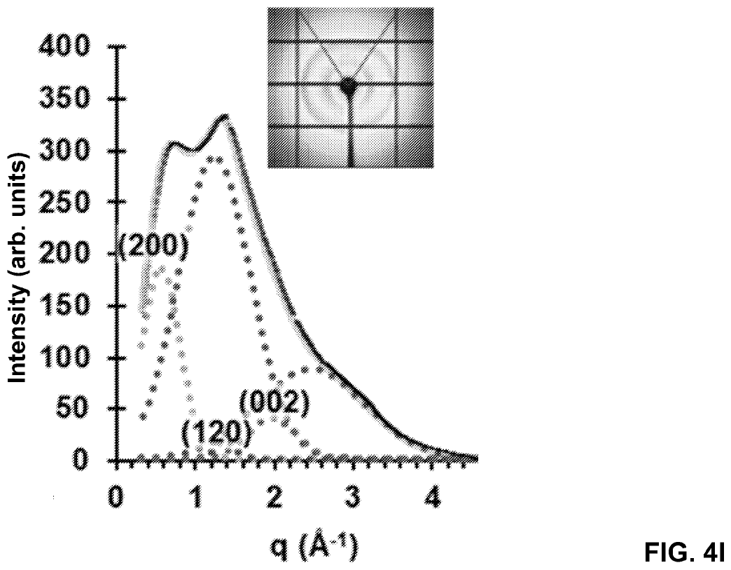

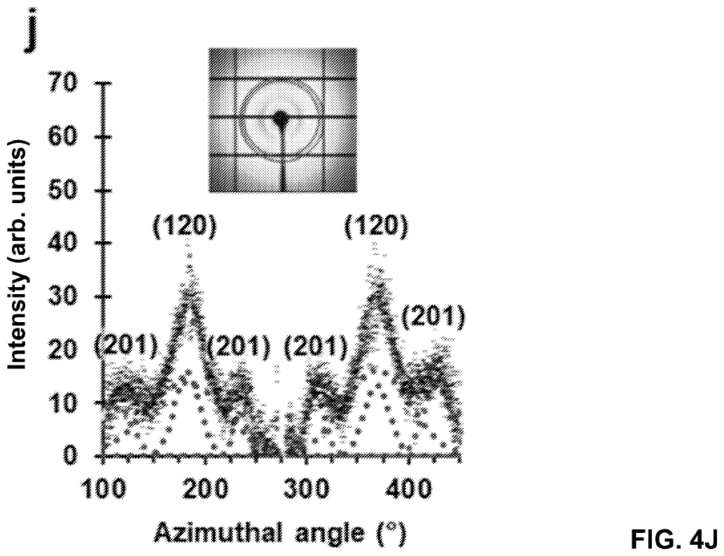

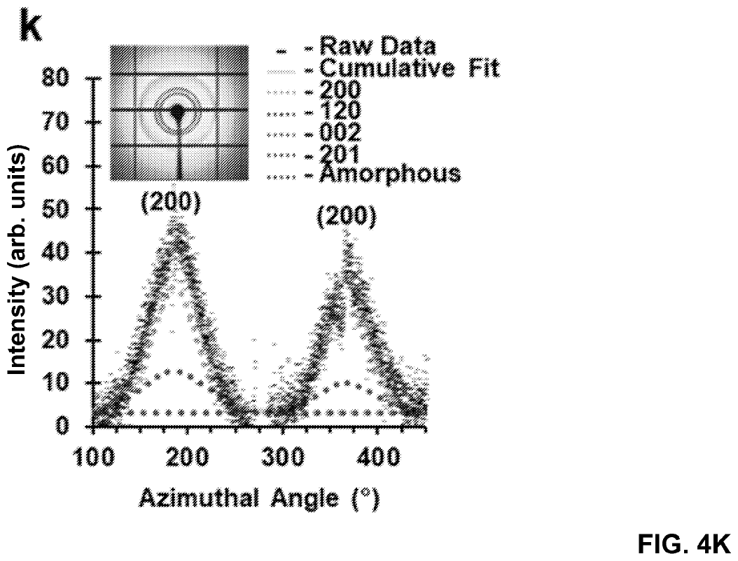

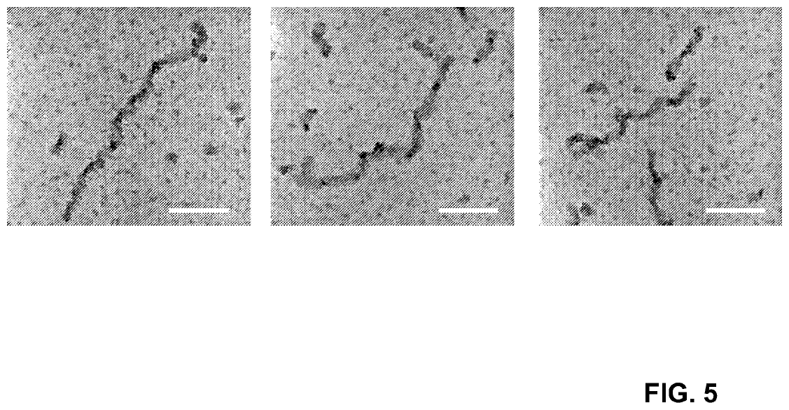

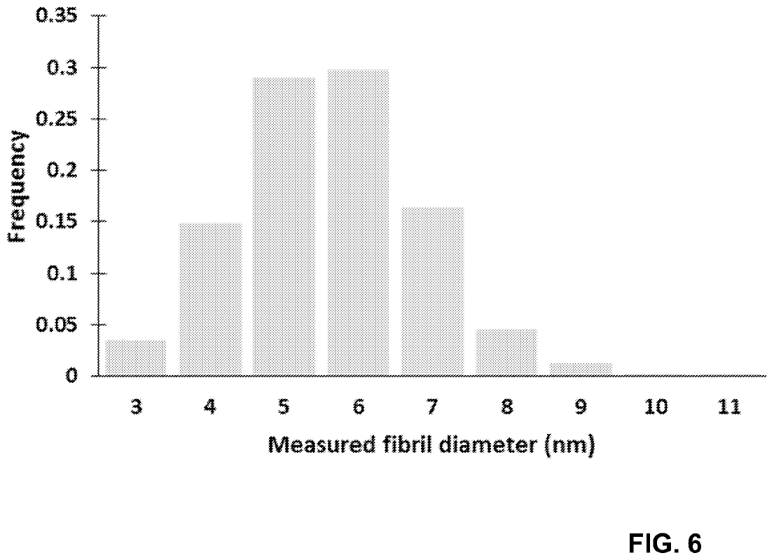

Circular dichroism (CD) was used to examine the secondary structure of the purified and refolded polymer, yielding spectra that are qualitatively similar to those previously reported for natural titin protein extracts (FIG. 4A). Further deconvolution and fold recognition by the BestSel deconvolution tool indicates a high degree of anti-parallel β-sheet structure (FIG. 4A, inset) and an immunoglobulin-like topology based on six of the top ten closest structures in the eight-dimensional BestSel secondary structural space (described herein below). Additionally, scanning transmission electron microscopy (STEM) of the purified titin polymer (FIG. 4B, FIG. 5) showed the presence of numerous nanoscale fibrils with apparent chain-of-beads structures and cross-sectional diameters (6.1+1.2 nm, FIG. 6) that were similar to those previously observed for natural titin proteins. Together, these results indicate that the microbial production system disclosed herein synthesizes UHMW titin polymers with a substantial degree of folded structures similar to natural Ig domains (hereafter called Ig-like structures).

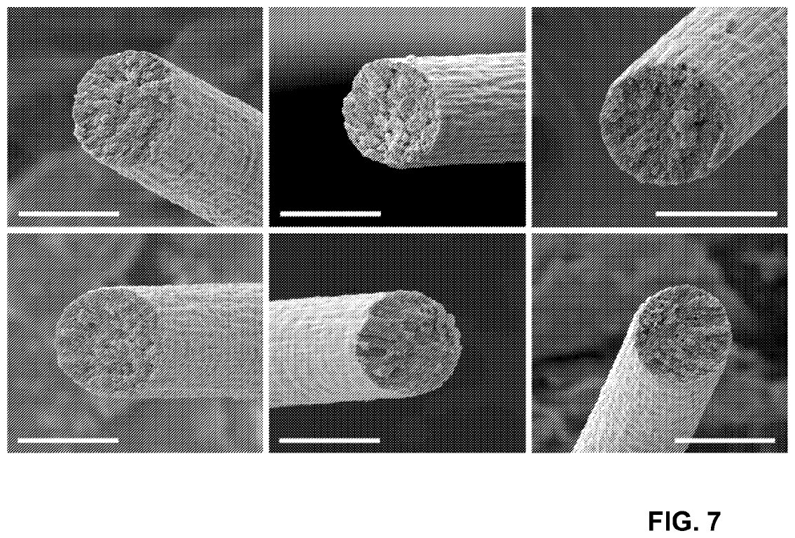

To explore practical uses of the microbially produced UHMW titin proteins disclosed herein, the polymer proteins were processed into macroscale monofilament fibers. It is known that networks of inter-chain crystallites, serving as non-covalent cross-links embedded in an amorphous matrix, provide high strength and toughness at the macroscale. If the polymers were refolded from a denatured state at high concentration, the robust folding of Ig domains favored the formation of a network of inter-chain β-sheet crystals. The titin polymer was dissolved at high concentration in the denaturing solvent hexafluoroisopropanol (HFIP) and then the resulting polymer dope solution was extruded through a narrow-bore needle into water with the aim of driving rapid titin refolding during fiber formation. Fibers were formed, which were subsequently subjected to post-spin draw to promote axial alignment of the expected crystalline domains. Both light microscopy and scanning electron microscopy (SEM) revealed cylindrical, monofilament fibers with highly consistent diameters of approximately 10 μm (FIG. 4C, FIG. 4D, FIG. 7, Table 1). Based on the observed spinning efficiency and protein yields, 1 L of shake flask batch culture is estimated to yield approximately 250 meters of fiber in a continuous spinning process.

TABLE 1
Measured diameters and mechanical properties of titin fibers made from microbially
produced UHMW titin. Clamp-to-clamp gauge length for all fibers was 5 mm.

| | | | | Avg. | | | | |
|---|---|---|---|---|---|---|---|---|
| | Diam. | Diam. | Diam. | Diameter | σ | E | ε | U_{T} |
| FIBER | A (μm) | B (μm) | C (μm) | (μm) | (MPa) | (GPa) | (%) | (MJ/m³) |
| 1 | 9.97 | 9.29 | 10.43 | 9.90 | 360 | 3.90 | 53 | 145 |
| 2 | 9.96 | 9.79 | 10.25 | 10.00 | 345 | 3.82 | 52 | 129 |
| 3 | 9.99 | 10.42 | 10.42 | 10.28 | 345 | 4.57 | 50 | 132 |
| 4 | 9.95 | 9.64 | 10.91 | 10.17 | 344 | 3.69 | 53 | 134 |
| 5 | 10.52 | 9.66 | 10.00 | 10.06 | 368 | 4.56 | 50 | 137 |
| 6 | 9.64 | 10.16 | 9.65 | 9.82 | 367 | 4.52 | 48 | 134 |
| 7 | 8.50 | 9.20 | 10.16 | 9.29 | 438 | 4.58 | 49 | 157 |
| 8 | 10.73 | 9.43 | 10.06 | 10.07 | 372 | 4.75 | 52 | 144 |
| 9 | 10.11 | 10.45 | 10.42 | 10.33 | 358 | 3.73 | 52 | 136 |
| 10 | 8.18 | 9.33 | 8.99 | 8.83 | 426 | 4.14 | 35 | 104 |
| 11 | 8.98 | 8.66 | 8.82 | 8.82 | 436 | 4.47 | 41 | 126 |
| 12 | 8.78 | 9.33 | 10.55 | 9.55 | 375 | 3.36 | 39 | 101 |
| 13 | 8.83 | 8.51 | 8.82 | 8.72 | 442 | 5.20 | 36 | 114 |
| 14 | 10.77 | 10.92 | 11.08 | 10.92 | 313 | 3.27 | 54 | 124 |

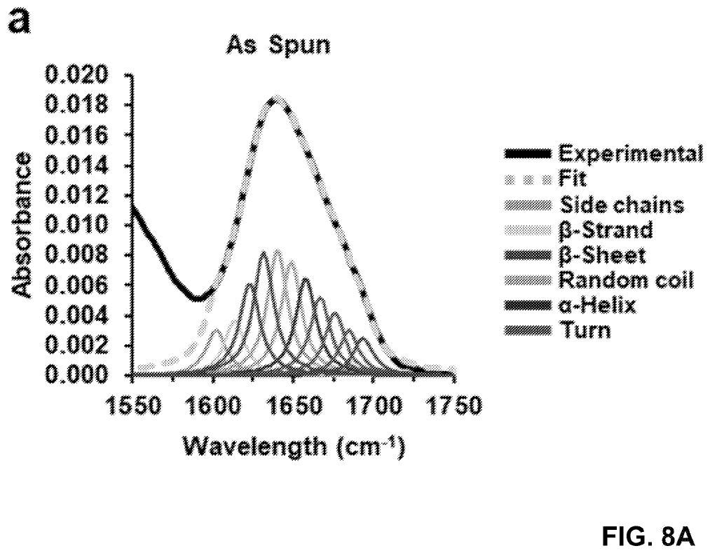

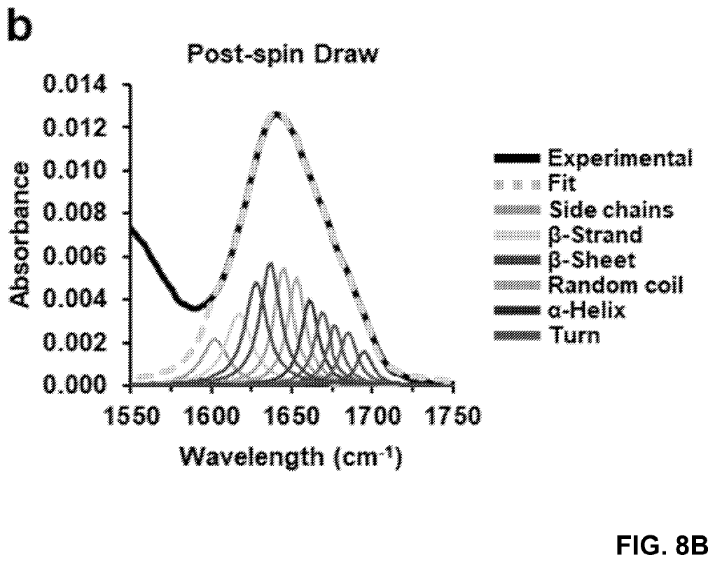

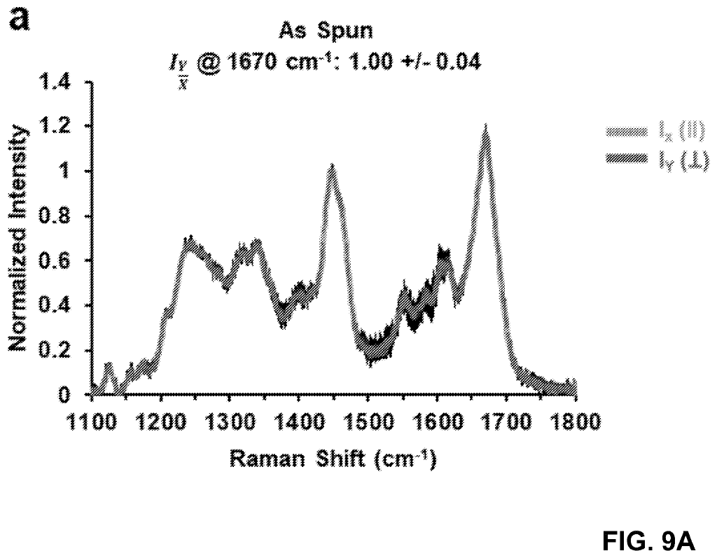

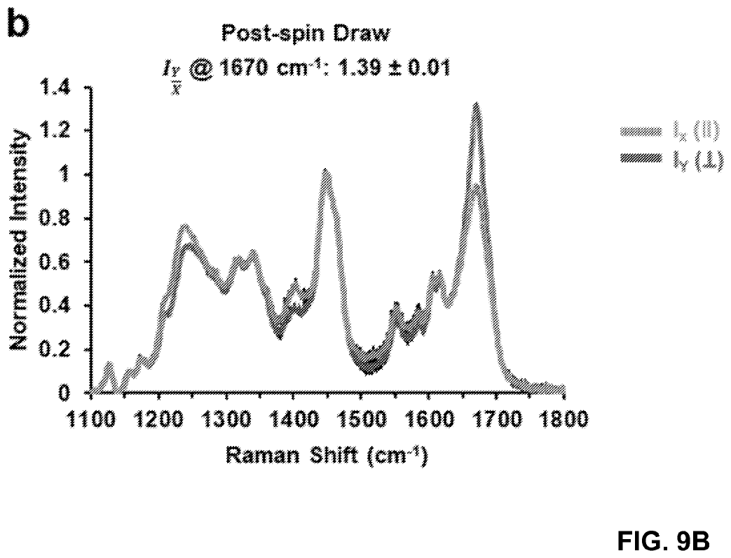

Fourier-transform infrared spectroscopy (FT-IR) analysis of the UHMW titin fibers confirmed a substantial percentage of β-sheet secondary structure in the fiber (FIG. 4F, FIGS. 8A and 8B). Deconvolution of the amide I peak allowed for estimation of a β-sheet content of approximately 28%, with no significant difference between fibers with or without post-spin draw (FIG. 4E, FIG. 4F). Polarized Raman spectromicroscopy was then used to examine alignment of β-sheets along the fiber axis. The as-spun fibers showed no orientation sensitivity in the amide I and II bands (FIG. 9A), indicating that the β-sheets formed during fiber spinning are randomly oriented. Conversely, polarized Raman spectra of the post-spin drawn fibers revealed a high degree of orientation sensitivity in the amide I and II bands, which is characteristic of axially aligned β-sheet structures (FIG. 4G, FIG. 9B). These results confirm that the spinning process produces a fiber that is rich in β-sheets with initially random orientation, while the subsequent post-spin draw helps to align these β-sheets along the fiber axis.

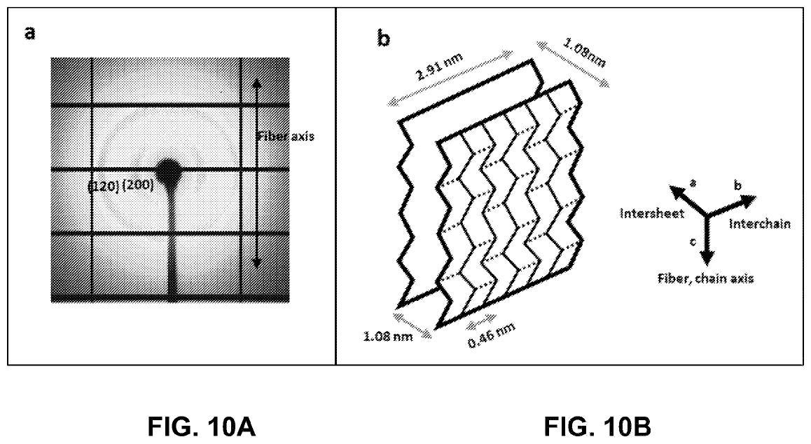

To further examine the structure of the post-drawn UHMW titin fibers, synchrotron-based wide-angle X-ray diffraction (WAXD) was employed. Two-dimensional diffraction images revealed two broad but distinct equatorial reflections perpendicular to the fiber axis, along with substantial amorphous components (FIGS. 4H-K, FIG. 10A), characteristic of a semi-crystalline material. The deconvolution of peaks indicates approximately 18% crystallinity (see Table 2 and Table 3). To examine the crystalline orientational order, the azimuthal 1D profiles of the two primary reflections were analyzed and determined an orientation parameter ƒ_{crystal}=0.76 for the crystalline portion of the titin fibers (see FIG. 4J, FIG. 4K, Table 2, Table 3). This indicates a substantial axial crystallite alignment, in agreement with the polarized Raman spectromicroscopy analysis (FIGS. 9A and 9B). Average crystallite size was estimated through further deconvolution of peaks and application of the Scherrer equation. In this manner, both the average crystallite size along the a-axis (inter-sheet axis) and the d-spacing were calculated to be 1.08 nm (see FIG. 4H, FIG. 4I, FIG. 10B, Table 2, and Table 3). This is in close agreement with the average distance (approximately 1.01 nm) between opposing β-sheets of individual Ig domains in the previously reported 167-170 crystal structure (FIG. 2). The fact that both the d-spacing and average crystallite size along the a-axis are 1.08 nm indicates that the titin fiber contains β-crystals of two β-sheets, similar to the structure of the native titin Ig domain. Meanwhile, average crystallite size along the b-axis (inter-strand axis) was calculated to be 2.91 nm, with an inter-strand d-spacing of 0.46 nm (FIG. 10B, Table 2, and Table 3). The observed inter-strand d-spacing in the titin fibers agrees with the average inter-strand distances of anti-parallel-strands in the crystal structure of 167-170 (approximately 0.46 nm, FIG. 2). However, the calculated b-axis width of 2.91 nm indicates an average of 6 β-strands per β-sheet, more than what is observed in the native titin Ig domains (3-4 β-strands per β-sheet, average width 1.46 nm, FIG. 2), indicating that some of the crystals form from side-by-side packing of pairs of Ig-like domains.

TABLE 2
Parameters extracted from the WAXD image of titin fiber
made from microbially produced UHMW titin protein.

| Mode of | | | Full Width at Half | |
|---|---|---|---|---|
| Integration | Peak Index | Peak Center (PC) | Maximum (FWHM) | Intensity (I) |
| Equatorial | (200) | 0.584 (Å⁻¹) | 0.523 (Å⁻¹) | 107 (a.u.) |
| | (120) | 1.37 (Å⁻¹) | 0.195 (Å⁻¹) | 8.73 (a.u.) |
| | Amorphous 1 | 1.25 (Å⁻¹) | 1.22 (Å⁻¹) | 376 (a.u.) |
| | Amorphous 2 | 2.36 (Å⁻¹) | 1.65 (Å⁻¹) | 158 (a.u.) |
| Meridian | (200) | 0.564 (Å⁻¹) | 0.565 (Å⁻¹) | 111 (a.u.) |
| | (120) | 1.37 (Å⁻¹) | 0.269 (Å⁻¹) | 5.13 (a.u.) |
| | (002) | 1.97 (Å⁻¹) | 0.717 (Å⁻¹) | 29.7 (a.u.) |
| | Amorphous 1 | 1.24 (Å⁻¹) | 1.07 (Å⁻¹) | 332 (a.u.) |
| | Amorphous 2 | 2.47 (Å⁻¹) | 1.60 (Å⁻¹) | 147 (a.u.) |
| Azimuthal | (120) | 183/370 (°) | 29.3/25.3 (°) | 497/240 (a.u.) |
| (120) | (201) | 115/235/312/426 | 34.3/15.6/22.6/35.1 | 336/138/252/528 |
| | | (°) | (°) | (a.u.) |
| | Amorphous | 181/363 (°) | 91.6/59.4 (°) | 1340/1300 (a.u.) |
| Azimuthal | (200) | 186/367 (°) | 53.6/54.2 (°) | 164/138 (a.u.) |
| (200) | Amorphous | 187/366 (°) | 80.4/70.3 (°) | 80.5/50.5 (a.u.) |

TABLE 3
Values calculated from WAXD diffraction parameters.

| Calculated | | |
|---|---|---|
| Parameter | Value | Calculation Method |
| a-axis (inter- sheet) D- spacing | 1.08 nm | $\frac{2 π}{P {C}_{Equatorial ( 200 )}} = \frac{2 π}{0.584 {Å}^{- 1}}$ |
| b-axis (inter- chain) D- spacing | 0.46 nm | $\frac{2 π}{P {C}_{Equatorial ( 120 )}} = \frac{2 π}{1.37 {Å}^{- 1}}$ |
| a-axis (inter- sheet) crystal size | 1.08 nm | $\begin{matrix}\frac{K \times λ}{arc sin ( \frac{2 \times λ \times {FWHM}_{Equatorial ( 200 )}}{4 π} ) \times cos ( arc sin ( \frac{λ \times {PC}_{Equatorial ( 200 )}}{4 π} ) \times \frac{360}{2 π} )} = \\ \frac{0.9 \times 0.886 Å}{arc sin ( \frac{2 \times 0.886 Å \times 0.523 {Å}^{- 1}}{4 π} ) \times cos ( arc sin ( \frac{0.886 Å \times 0.584 {Å}^{- 1}}{4 π} ) \times \frac{360}{2 π} )}\end{matrix}$ |
| b-axis (inter- chain) crystal size | 2.91 nm | $\begin{matrix}\frac{K \times λ}{arc sin ( \frac{2 \times λ \times {FWHM}_{Equatorial ( 120 )}}{4 π} ) \times cos ( arc sin ( \frac{λ \times {PC}_{Equatorial ( 120 )}}{4 π} ) \times \frac{360}{2 π} )} = \\ \frac{0.9 \times 0.886 Å}{arc sin ( \frac{2 \times 0.886 Å \times 0.195 {Å}^{- 1}}{4 π} ) \times cos ( arc sin ( \frac{0.886 Å \times 1.37 {Å}^{- 1}}{4 π} ) \times \frac{360}{2 π} )}\end{matrix}$ |
| % Crystallinity | 18% | $\begin{matrix}\frac{{I}_{Equtorial ( 200 )} + {I}_{Equtorial ( 120 )}}{{I}_{Equtorial ( 200 )} + {I}_{Equtorial ( 120 )} + {I}_{Equtorial Amorphous 1} + {I}_{Equtorial Amorphous 2 )}} = \\ \frac{107 + 8.73}{107 + 8.73 + 376 + 158}\end{matrix}$ |
| f_{crystal} | 0.76 | ${f}_{c r y s t a l} = \frac{3 {cos}^{2} φ - 1}{2}$ |
| | | $\begin{matrix}{cos}^{2} φ = 1 - 0.8 \times {sin}^{2} ( 0.4 \times F W H {M}_{E q u a torial ( 200 )} ) - 1.2 \times {sin}^{2} ( 0.4 \times F W H {M}_{E q u a torial ( 120 )} ) = \\ 1 - 0. 8 \times {sin}^{2} ( 0.4 \times \frac{53.6 + 54.2}{2} \times \frac{2 π}{360} ) - 1.2 \times {sin}^{2} ( 0.4 \times \frac{29.3 + 25.3}{2} \times \frac{2 π}{360} )\end{matrix}$ |

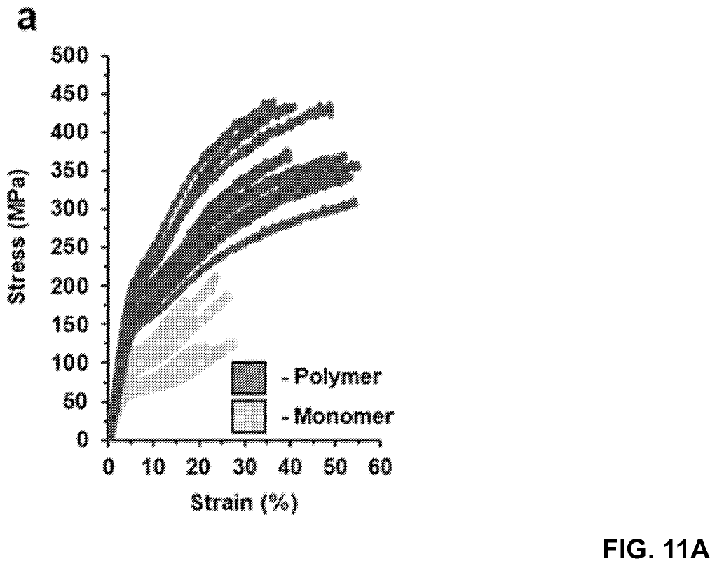

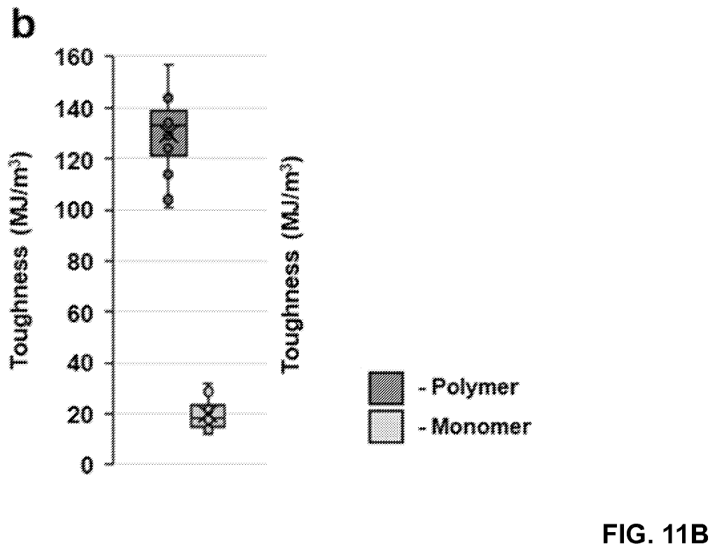

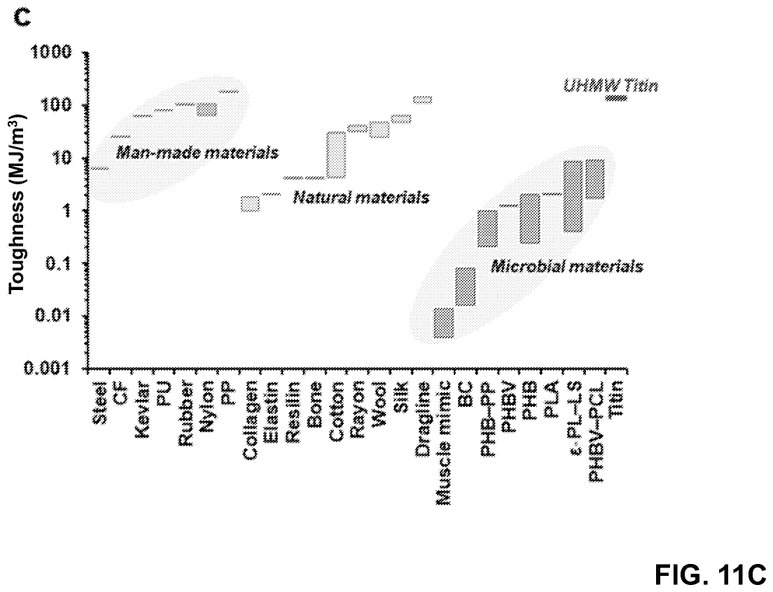

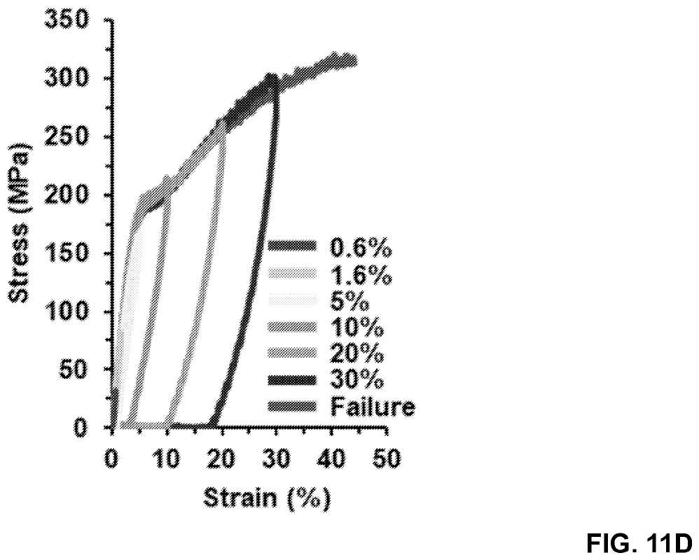

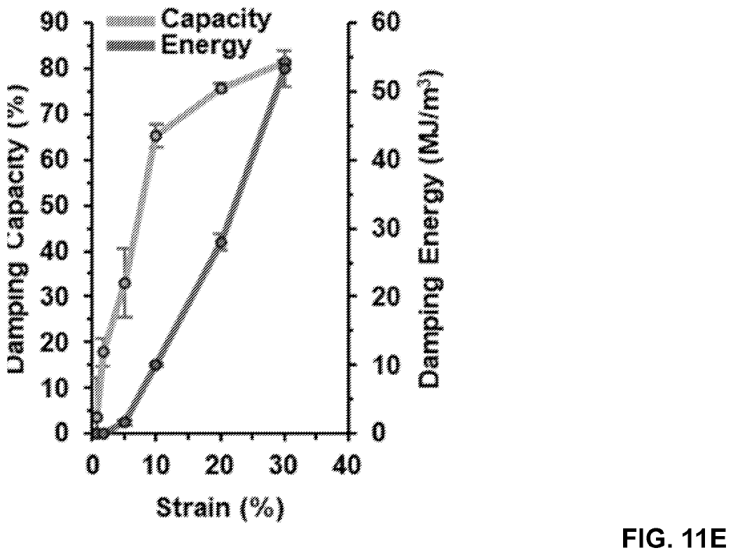

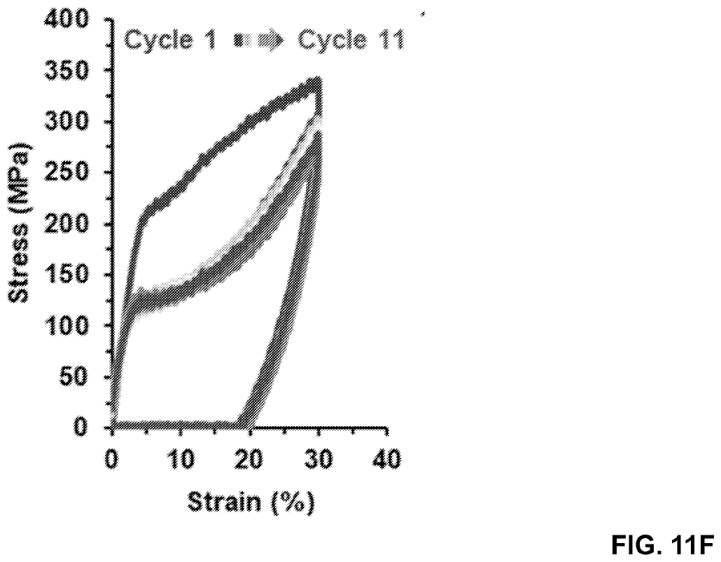

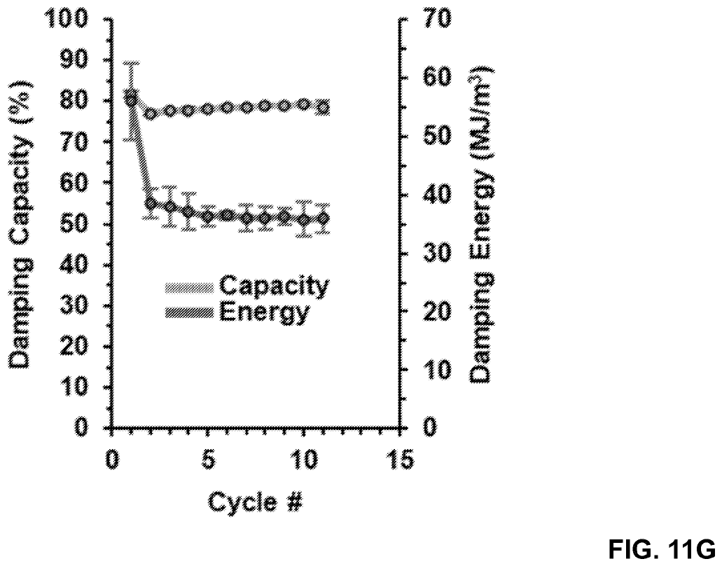

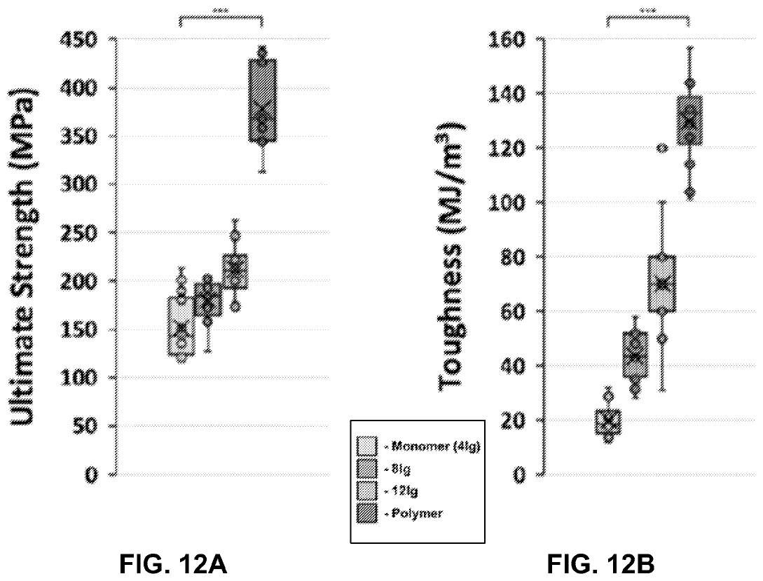

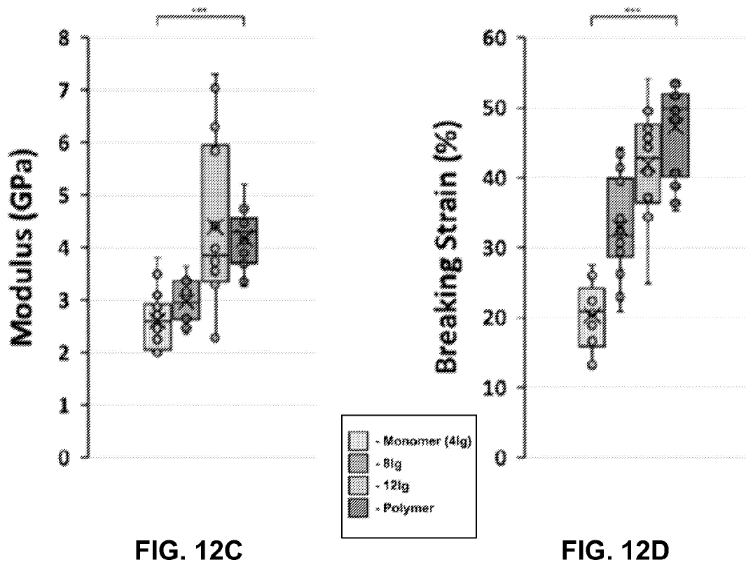

The mechanical properties of the titin fibers made from microbially produced UHMW titin were next investigated to determine whether they reproduce the desirable mechanics of natural muscle fibers. Tensile testing revealed exceptional strength (378±41 MPa), modulus (4.2±0.6 GPa), extensibility (47±7%), and toughness (130±15 MJ/m³) (FIGS. 11A, 11B, 11C, and FIGS. 12A, 12B, 12C, and 12D). Both the strength and toughness of these fibers far exceed values measured for muscle fibers and individual myofibrils. Furthermore, these toughness measures even exceed those of many of the toughest synthetic and natural materials, and far exceed those of traditional microbial materials (FIG. 11C). SEM images of fibers after fracture indicate a uniform, densely packed microscale morphology (FIG. 7). Meanwhile, fibers spun in an identical manner from the low MW 4Ig exhibited dramatically lower strength (−60%), modulus (−38%), breaking strain (−57%), and toughness (−85%), when compared to the UHMW fibers (FIG. 11A, FIG. 11B, and FIGS. 12A-D). This result confirms that the high mechanical performance of the fibers partially results from the previously unobtainable UHMW of the microbially produced titin polymer, highlighting the value of the protein polymerization strategy.

While it is generally accepted that the MW of a polymer can greatly affect the mechanical properties of the resulting material, the present disclosure provides further evidence for a correlation in titin fibers and to confirm that the MW was a primary factor in the observed performance of the UHMW titin fibers. To produce materials of a precise MW, genes that contained two and three repeats of the 4 Ig sequence were constructed, referred to as 8 Ig and 12 Ig, respectively. These proteins were then expressed, purified, and spun into fibers. Tensile testing of these fibers revealed a strong positive correlation between MW and mechanical properties (FIGS. 12A-D). Still, the UHMW fibers offered far greater strength and toughness (79% and 85%, respectively) than the 12 Ig fibers, while eliminating risks of genetic instability that could otherwise diminish bioproduction yields, further demonstrating the value of the in vivo polymerization platform.

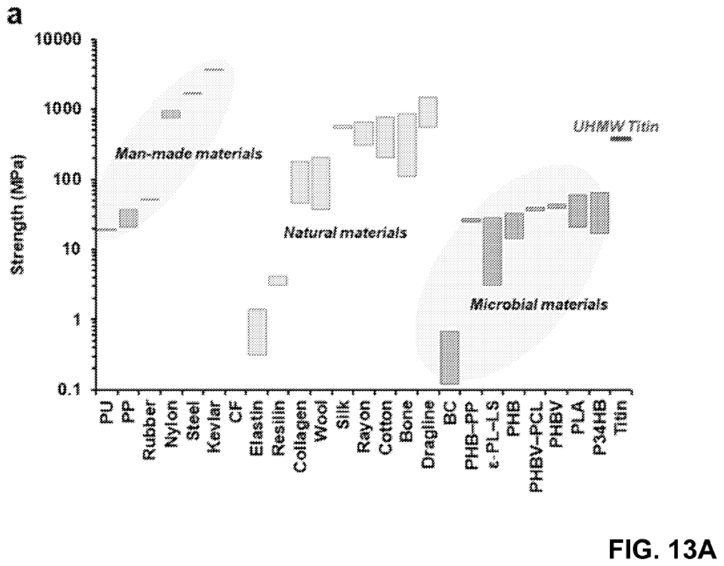

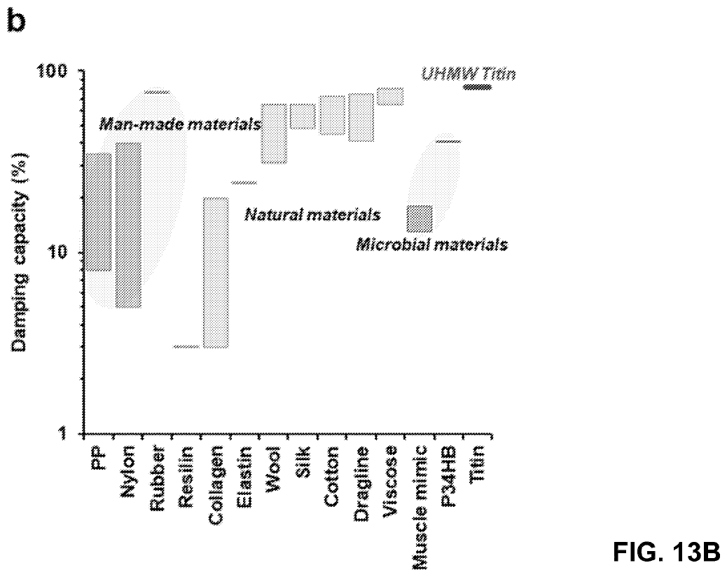

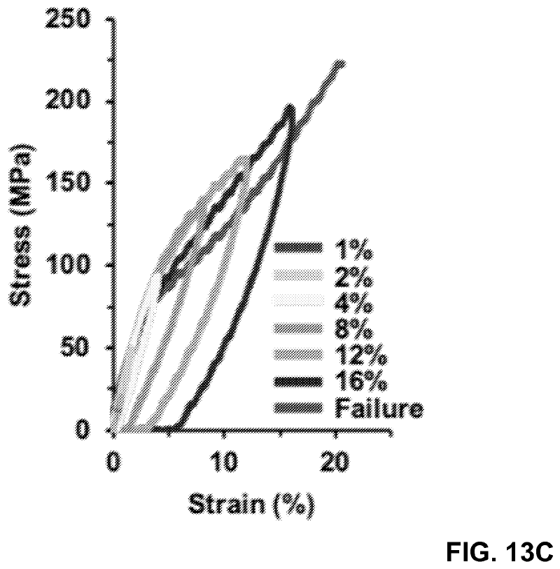

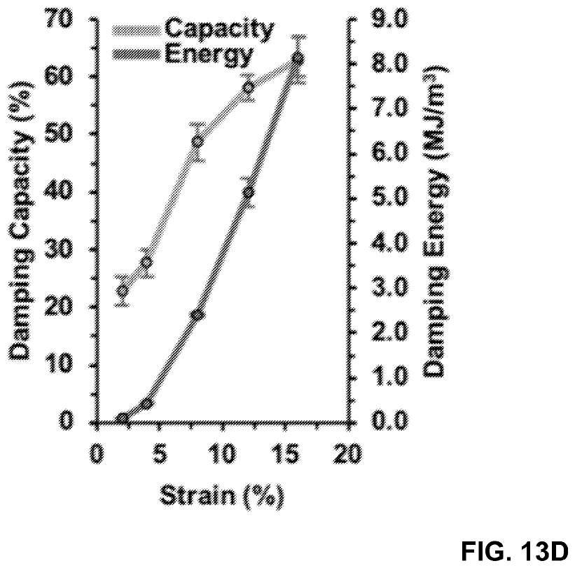

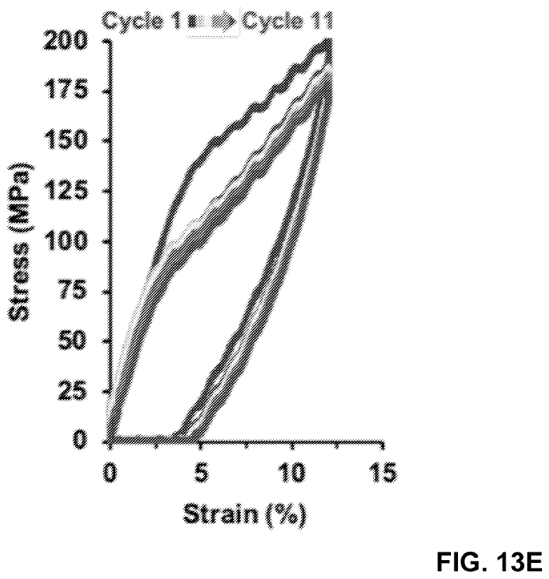

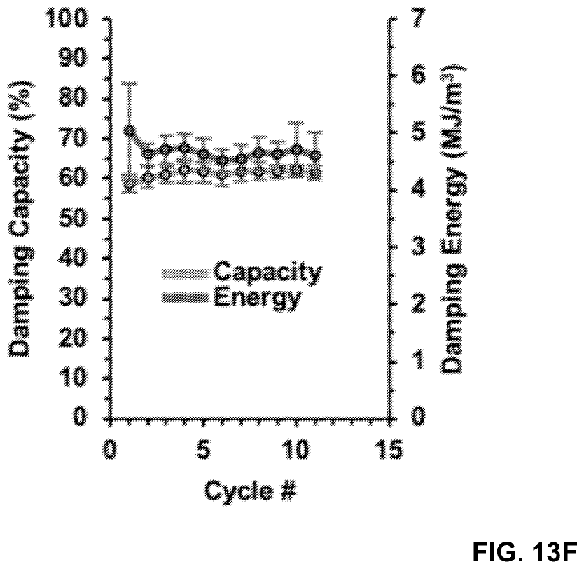

Within muscle fibers, the natural titin protein behaves as a resilient material at low strain, capable of reversible deformation without energy loss, and as an energy damping material at higher strain, capable of dissipating energy to prevent myofibril damage due to over-extension. This combination of and transition between resilient and energy damping states makes titin uniquely suited for the crucial biological functions of skeletal and cardiac muscle. To investigate whether the titin fibers made from microbially produced UHMW titin recapitulate this desirable combination of properties, cyclic force loading experiments were performed over a range of increasing strains. Up to 1.6% strain, the fibers are stretched elastically with relatively high resilience and low damping capacity (17.9±3.0%) and damping energy (0.1±0.0 MJ/m³) (FIG. 11D and FIG. 11E). However, as fiber extension was increased beyond 1.6%, damping capacity increased rapidly, reaching up to 81.3±0.4% at 30% strain (FIG. 11D and FIG. 11E). As expected, damping energy also rapidly increased with elongation, reaching a maximum of 53.3±2.6 MJ/m³ at 30% strain. This damping capacity exceeds previous measures of the damping capacity of both myofibrils and natural single molecule titin, which have been measured at around 60%, as well as many high-damping synthetic and natural materials (FIGS. 13A and 13B). When tested at increasing strains, fibers made from the 4Ig oligomers also demonstrated resilience at low strains followed by a rapid increase in damping capacity as the strain increased: however, the damping energy measured for the oligomer fibers was greatly reduced relative to those of the polymer fibers likely due to the decreased MW of the constituent proteins (FIGS. 13C and 13D).

During relaxation of natural single molecule titin after high extension, unfolded Ig domains are believed to refold, enabling a form of self-repair and recovery of mechanical properties. To examine whether the titin fiber made from the microbially produced UHMW titin disclosed herein has similar properties, the fibers were subjected to repeated rounds of loading-unloading cycles approaching the fiber breaking strain. After drawing fibers to 30% strain at ambient humidity (45% RH) and relaxing back to 0% strain, an apparent permanent set was observed at approximately 20% strain (i.e., fibers only rapidly recovered about 33% of the total deformation) (FIG. 11F, including Cycle 1). Consequently, to better mimic the aqueous environment of natural titin in the muscle, the stretched fibers were briefly exposed to high humidity air (95% RH) following relaxation, whereupon the fibers were observed to rapidly contract back to their original lengths (0% strain). Under these conditions, the second round of loading-unloading exhibited only a slight, 5% decrease in damping capacity and a 15 MJ/m³ decrease in damping energy compared to the first cycle (FIGS. 11F and 11G). Additional loading-unloading cycles resulted in no further reduction of damping capacity and only an additional 3 MJ/m³ decrease in damping energy over a successive 10 cycles (FIGS. 11F and 11G). Fibers made from the 4Ig titin oligomers also demonstrated high damping capacity (˜60%) and humidity-driven recovery when pulled to a near maximal strain (in this case 12%) over several cycles of loading, unloading, and humidity treatment (FIGS. 13E and 13F). Despite the damping energy of the 4Ig oligomer fibers being much lower (˜4.7 MJ/m³) due to differences in MW, in some embodiments the relatively high damping capacity indicates that the humidity-driven recovery of the fibers is mediated by the refolding of Ig domains. Thus, with 95% RH treatment, the titin fiber made from the microbially produced UHMW titin rapidly recover mechanical properties in a manner reminiscent of natural titin and muscle fiber. This intriguing regenerative behavior, along with the material's combination of exceptional damping capacity, strength, and toughness, indicate a broad range of potential applications for these fibers in areas such as anti-ballistic materials, netting, sutures, and tissue engineering.

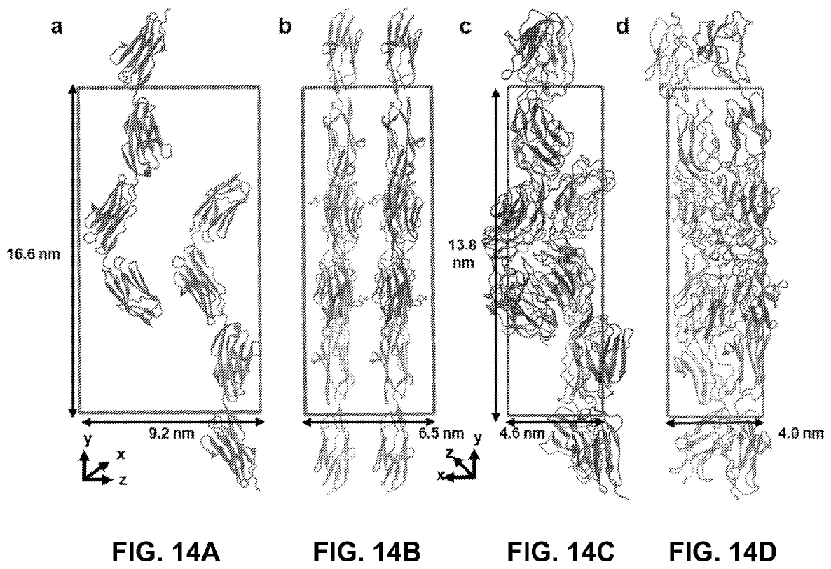

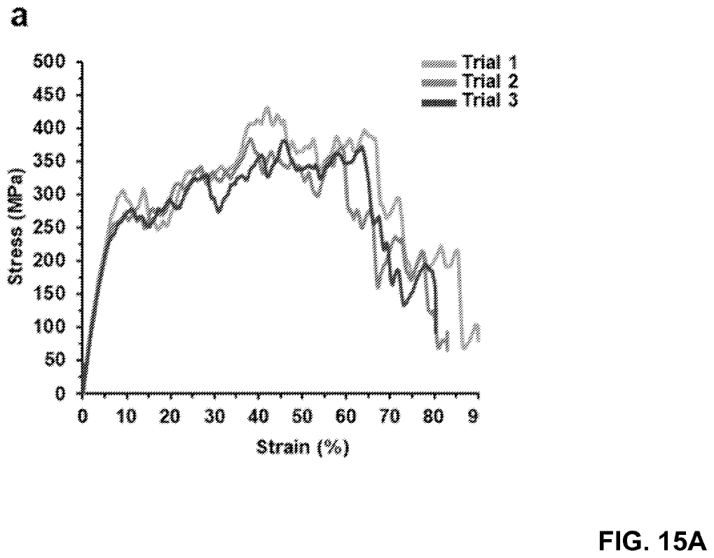

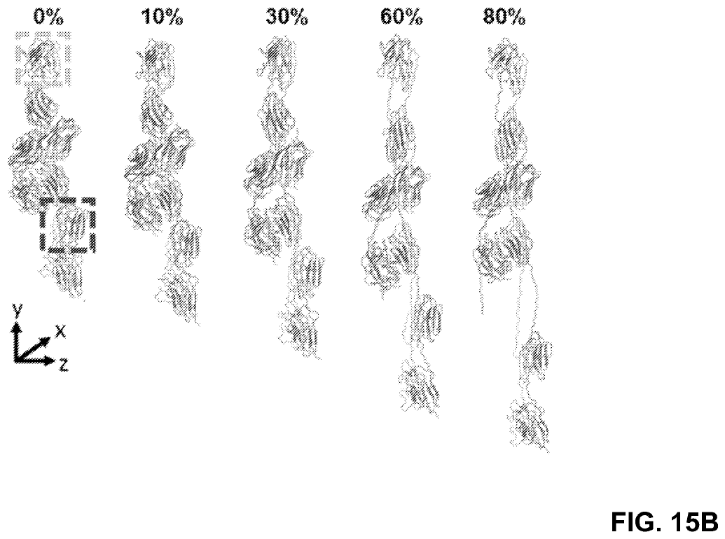

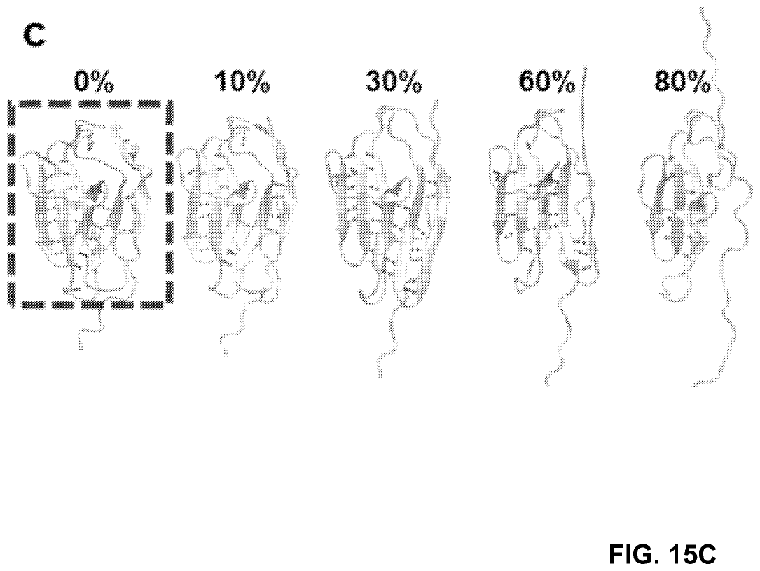

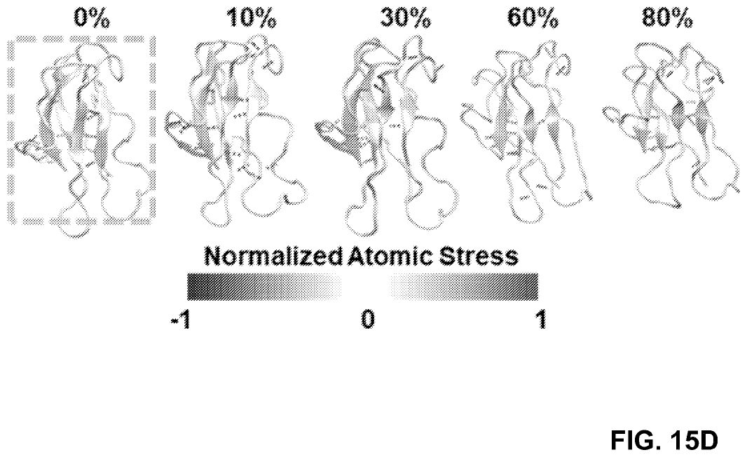

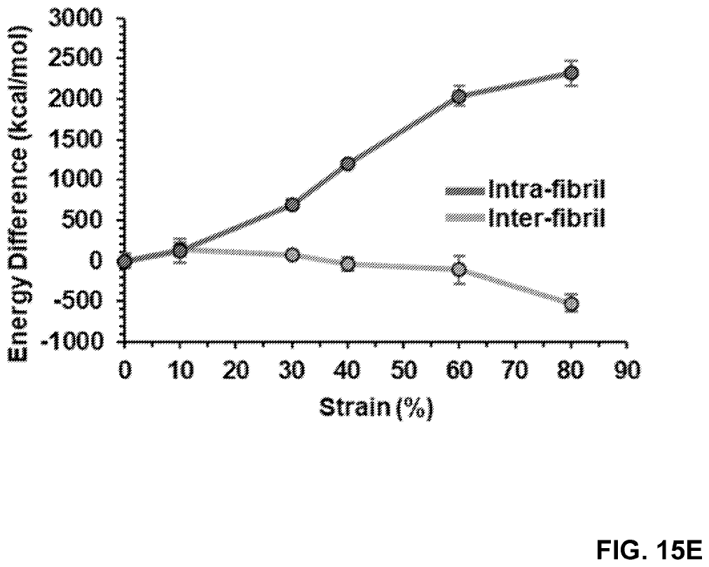

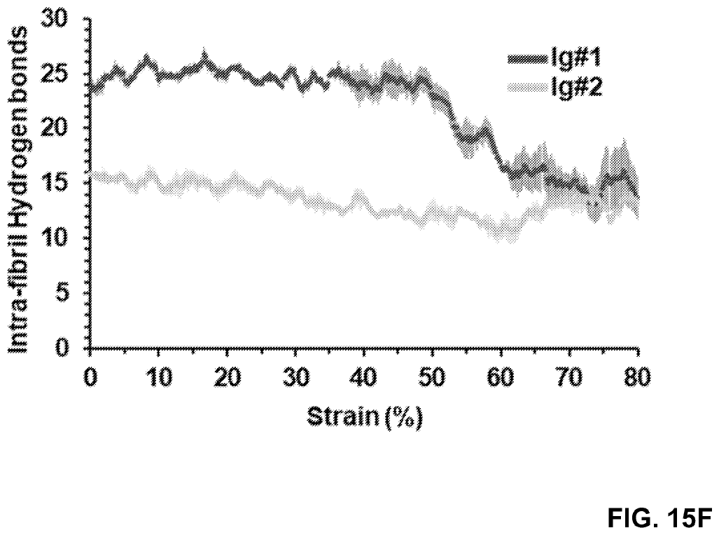

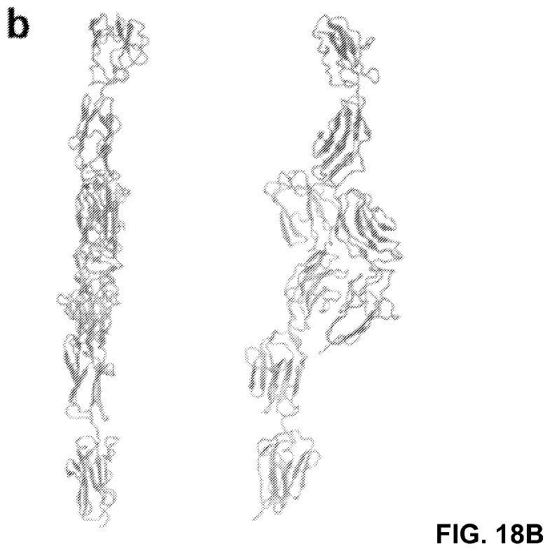

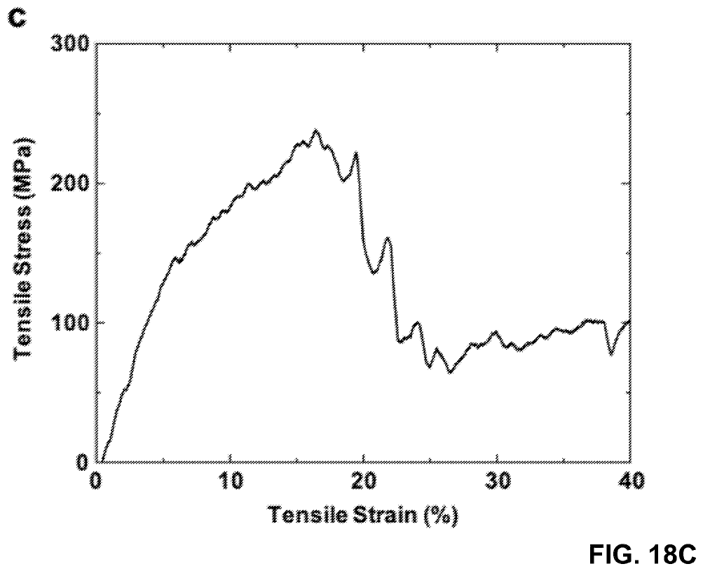

To help elucidate the possible energy dissipation mechanisms that result in the observed mechanical properties of the UHMW titin fibers, molecular dynamics (MD) simulations were carried out to examine the behavior of modeled titin fibers under tensile deformation. While the stretching behavior of single titin chains has been widely studied, both with AFM experiments and steered MD (SMD) simulations, the macroscale biosynthetic titin fiber described herein required a unique model featuring a network of tightly packed titin chains and periodic boundary conditions to provide a better representation of the bulk properties. An initial fiber molecular model was built according to the results of structural analyses, which indicated that pairs of Ig-like domains from adjacent titin polymer chains are packed side-by-side and axially aligned (see FIGS. 14A-D and FIGS. 18A-C). A uniaxial tensile strain was then applied to the simulated fiber and a tensile stress-strain curve was measured. The resulting modeled curves agreed well with the experimental results (FIG. 15A), showing an initial elastic deformation up to approximately 5% strain, with modulus of 3.6±0.2 GPa (compared to 4.2±0.6 GPa for the experimental titin fibers), followed by a period of plastic deformation and strain hardening with a peak stress of 378±17 MPa (compared to 378±41 MPa for the experimental titin fibers). While the real fibers exhibited a sudden failure at an average strain of 47%, the model showed a relatively gradual decrease in stress beyond 45%. This difference is attributed to the size effects of the small volume of the MD simulation relative to that of the experimental specimen. Specifically, in the macroscale fibers, the presence of defects and strain localization upon yielding are expected to result in a more sudden failure than that observed at the molecular scale.

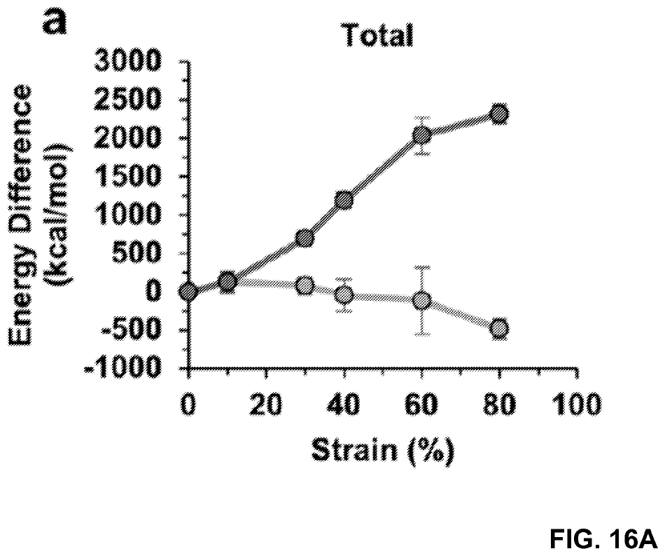

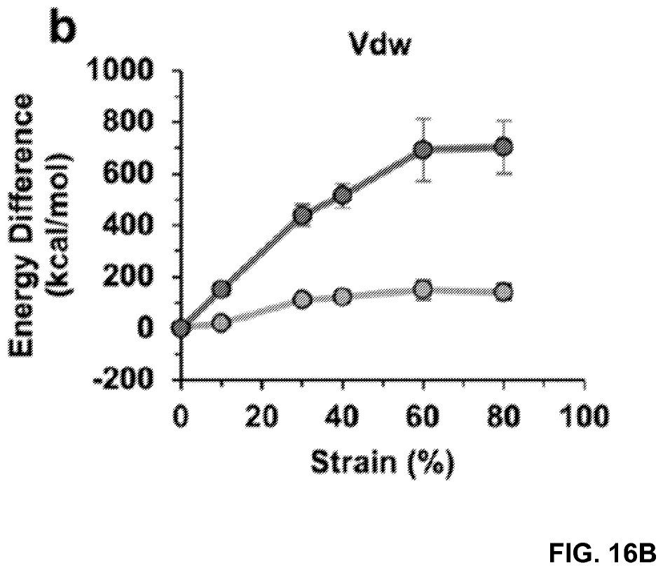

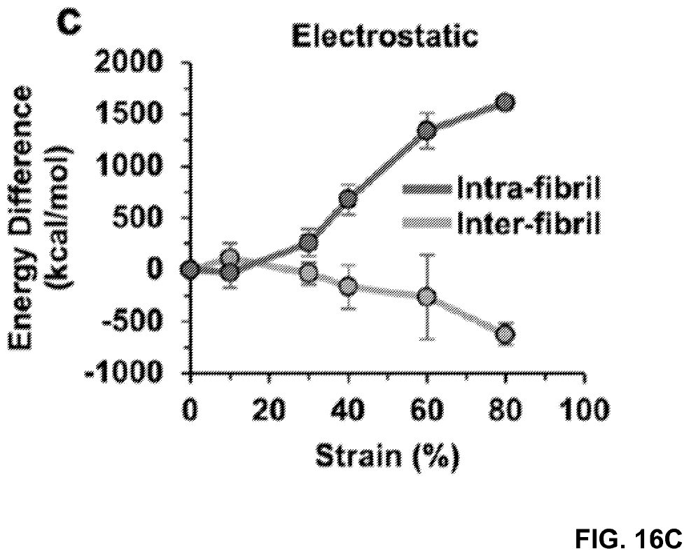

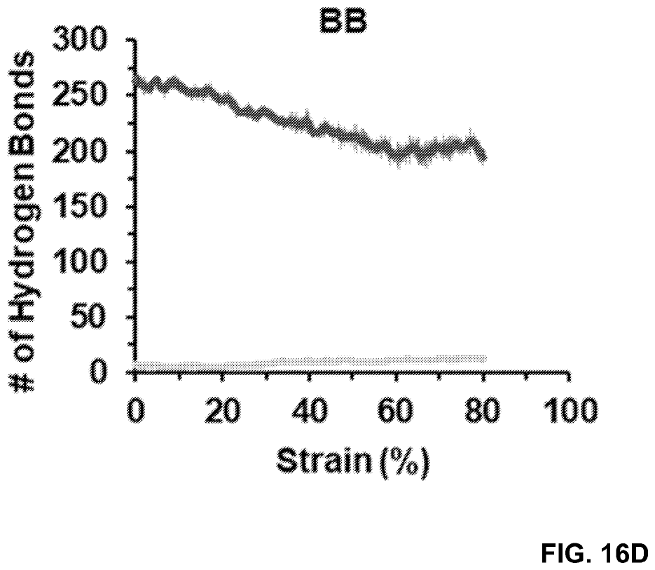

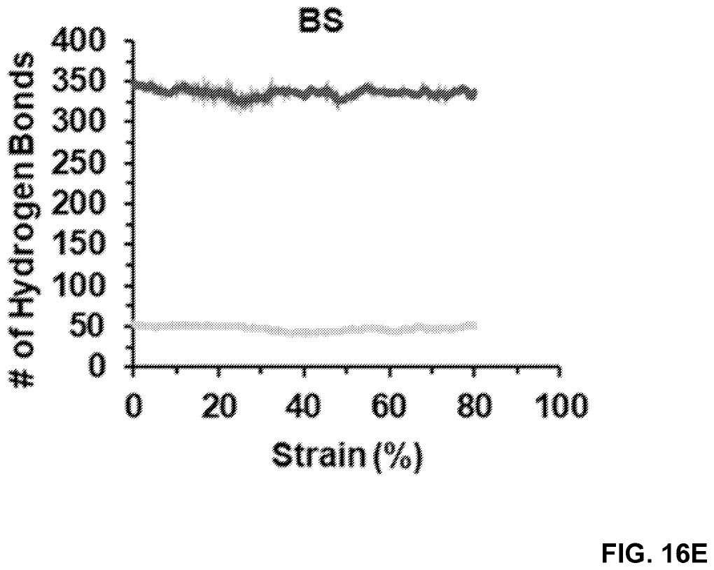

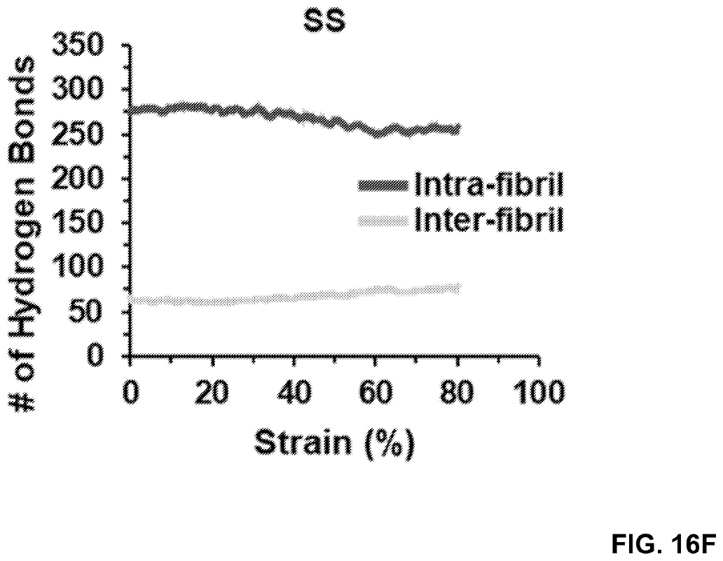

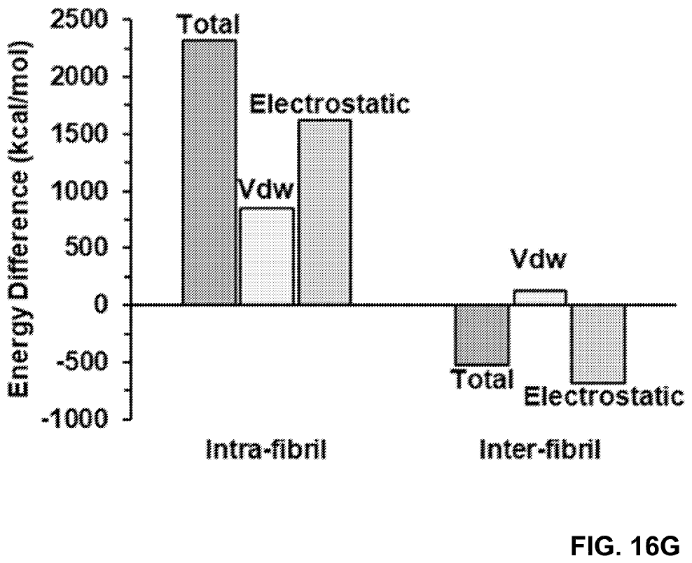

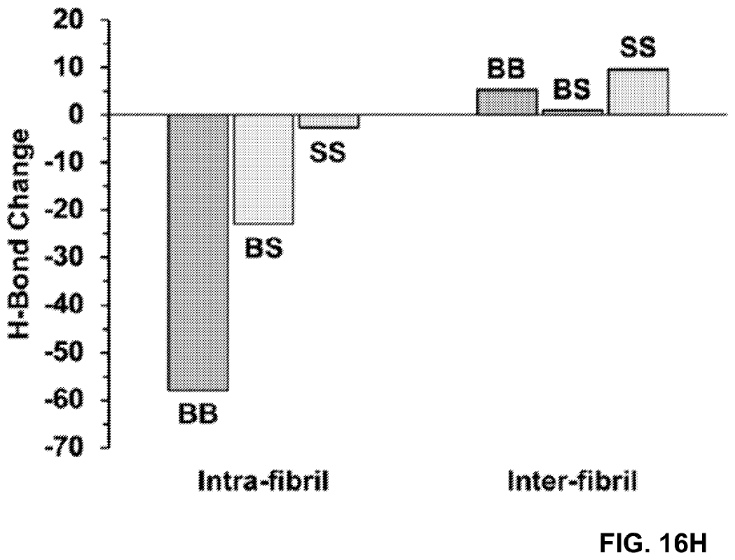

Observation of structural and energy changes throughout the course of the simulation indicates that up to 10% strain there is little change in the structure or relative position of Ig domain pairs (FIG. 15B), with tensile stress distributed evenly across the structure (FIG. 15C and FIG. 15D). While there is a slight disruption of intra-fibril Van der Waals interactions in this regime (FIGS. 16A and 16B), there is no substantial change in electrostatic interactions or hydrogen bonding (FIGS. 16C, 16D, 16E, and 16F). In the subsequent plastic deformation and strain-hardening regime (10-50% strain), the linker regions between Ig domains become fully extended (FIG. 15B) and stress is accumulated within Ig-like domains (FIG. 15C and FIG. 15D), disrupting intra-fibril hydrogen bonds (primarily backbone-backbone, FIGS. 16D, 16E, and 16F) and electrostatic interactions (FIG. 16C) in addition to continued disruption of intra-fibril Van der Waals interactions (FIG. 16B). Interestingly, throughout this regime, overall inter-fibril interactions actually increase (FIG. 15E) driven by increases in inter-fibril electrostatic interactions (FIG. 16C) and inter-fibril hydrogen bonding (FIGS. 16D, 16E, 16F, 16G, and 16H), indicating that fiber pulling actually drives annealing of the inter-fibril pairs of Ig-like domains, further strengthening the fibers. Finally, at high strains (50-80%), some Ig-like domains undergo substantial unfolding (FIG. 15B and FIG. 15C), with a major disruption of intra-fibril hydrogen bonding within these sacrificial domains (FIG. 15F, red line). Remarkably, this unfolding occurs before any apparent slippage between adjacent titin polymer chains (FIG. 15B), with continued increase in overall inter-fibril interactions (FIG. 15E) including inter-fibril electrostatic interactions and hydrogen bonding (FIGS. 16C, 16D, 16E, and 16F). Furthermore, due to the unfolding of the sacrificial Ig domains, stress is reduced in other Ig domains, allowing them to relax and regain some stabilizing intramolecular hydrogen bonding (FIG. 15D and FIG. 15F, yellow line). While unfolding is observed in the model above 50% strain, such Ig domain unfolding occurs within the macroscale fiber at lower strains in areas of concentrated stress, thus contributing to the overall energy damping and toughness of the macroscale titin fibers. Together, these modeling results indicate that the outstanding mechanical properties of the titin fibers made from microbially produced UHMW titin originate from a unique inter-fibril pairing of folded Ig-like domains. Such inter-chain non-covalent crosslinking through folded, stretchable domains has rarely been explored in either organic polymeric materials or other fibers made from microbially produced material.

By harnessing the biosynthetic power of microbes, a novel high performance material is described herein that recaptures not only the most desirable mechanical properties of natural muscle fibers (i.e., high damping capacity and rapid mechanical recovery) but also exceptionally high strength and toughness, superior even to many manmade and natural high-performance fibers. This is the first example of an engineered macroscale material produced from titin. The fiber's outstanding combination of mechanical properties, sustainable production process, and biodegradability make it an excellent candidate for environmentally friendly applications in a range of fields from biomedicine to commercial textiles (e.g., anti-ballistic materials, netting, sutures, and tissue engineering). Structural analyses indicate that these UHMW titin fibers contain axially aligned, side-by-side pairs of Ig-like domains. Molecular dynamics simulations indicate that such a network of non-covalent cross-linking through Ig-like domains strongly resists chain slippage, while permitting domain unfolding, giving rise to the observed combination of mechanical properties. Materials production through such non-covalent crosslinking of folded, stretchable proteins has rarely been explored and these results inform the design of other high-performance materials that exploit this paradigm to yield a range of intriguing macroscale material properties. The range of products accessible through engineered microbial synthesis as described herein, moving from primarily small molecules, peptides, therapeutic proteins, and industrial enzymes toward effective direct production of high-performance materials, represents a significant expansion. The biosynthetic strategies developed herein are applicable to other proteins with robust folding properties, yielding novel, high-performance materials with an expanded range of properties and offering an increasing variety of sustainable alternatives to traditional petroleum-based polymers.

### Exemplary Materials and Methods

The following materials and methods are exemplary in nature, and the present disclosure is not limited the specific materials and methods described in this section.

Strains and growth conditions. E. coli NEB 10-beta (NEB10β) was used for all plasmid cloning and protein production. For all cloning, E. coli strains were cultured in Terrific Broth (TB) containing 24 g/L yeast extract, 20 g/L tryptone, 0.4% v/v glycerol, 17 mM KH₂PO₄, and 72 mM K₂HPO₄ at 37° C. with appropriate antibiotics (50 μg/mL kanamycin). M9 glucose medium with tryptone supplement (2% w/v glucose, 1×M9 Salts, 75 mM MOPS pH 7.4, 12 g/L tryptone, 5 mM sodium citrate, 2 mM MgSO₄·7H₂O, 100 μM FeSO₄·7H₂O, 100 μM CaCl₂·2H₂O, 3 μM thiamine, 1× micronutrients [40 μM ZnSO₄·7H₂O, 20 μM CuSO₄·5H₂O, 10 μM MnCl₂·4H₂O, 4 μM H₃BO₃, 0.4 μM (NH₄)₆Mo₇O₂₄·4H₂O, and 0.3 μM CoCl₂·6H₂O]) was used for protein production in bioreactors.

Chemicals and reagents. Unless otherwise noted, all chemicals and reagents were obtained from MilliporeSigma. Plasmid purification and gel extraction kits were purchased from iNtRON Biotechnology. FastDigest restriction enzymes and T4 DNA ligase were purchased from Thermo Fisher Scientific and used for all digestions and ligations following manufacturer protocols.

Construction and expression optimization of titin oligomers and polymerization cassettes. The amino acid sequence of rabbit soleus titin domains 167-70 (SEQ ID NO. 1) was obtained from a recent publication, and the coding sequence was optimized for E. coli expression (Table 4). The resulting optimized sequence was synthesized as a gBlock fragment by Integrated DNA Technologies. The sequence was then inserted between the KpnI and Kpn2I restriction sites of a modified BglBricks vector containing gp41-1^{C} and gp41-1^{N} SIs under control of a PBAD promoter or a P_{LacO1} promoter, yielding plasmids p-1-4XT-1B and p-1-4XT-IL, respectively (Table 5). Additionally, the optimized titin sequence was inserted between the KpnI and Kpn2I restriction sites of a modified BglBricks vector containing no SIs under control of a PLacol promoter, yielding plasmid p-4XT (Table 5). To construct the 8Ig titin plasmid, PCR was first used to amplify the optimized 4XT sequence from p-4XT, adding a Kpn2I restriction site to the 5′ end and maintaining a stop codon and a BamHI site at the 3′ end of the amplicon (Table 6). This amplicon was then inserted downstream of the 4XT sequence in p-4XT via restriction digest and a two-part ligation, creating the p-8XT plasmid with the 4XT sequence duplicated (Table 5). The 12Ig plasmid (p-12XT) was made in a similar fashion. First, the 4XT sequence was PCR-amplified twice, once with primers adding a Kpn2I site to the 5′ end and a SpeI site to the 3′ end; and another time with primers adding a NheI site to the 5′ end and maintaining a stop codon and a BamHI site at the 3′ end (Table 6). The resulting PCR amplicons and p-4XT were digested with the corresponding restriction enzymes and ligated in a three-part reaction, yielding the p-12XT plasmid with a triplicated 4XT sequence (Table 5).

TABLE 4
Gene sequences.

| Sequence | | |
|---|---|---|
| Name | Description | Sequence |
| 4XT | Codon | GGTACCCACCACCATCACCATCATCCGCCGTTCTTCGATC |
| (SEQ ID | optimized | TGAAGCCGGTGTCTGTTGACCTGGCTCTGGGTGAATCTGG |
| NO. 1) | CDS for | CACCTTCAAATGCCACGTGACCGGCACCGCTCCGATCAA |
| | rabbit soleus | GATTACGTGGGCGAAAGACAACCGCGAGATTCGTCCAGG |
| | titin domains | CGGCAACTACAAAATGACCCTGGTGGAAAACACTGCGAC |
| | I67-I70 | CCTGACCGTCCTGAAAGTGACCAAAGGTGACGCGGGCCA |
| | | GTACACTTGTTACGCAAGCAACGTGGCGGGCAAAGATTC |
| | | TTGCTCTGCACAGCTGGGCGTGCAGGAACCGCCTCGCTTC |
| | | ATTAAAAAGCTGGAGCCGTCCCGCATCGTGAAACAGGAC |
| | | GAACATACCCGTTATGAATGCAAAATTGGCGGCTCTCCTG |
| | | AAATCAAAGTACTGTGGTATAAGGACGAAACCGAGATCC |
| | | AGGAGTCTTCTAAGTTCCGTATGTCTTTCGTGGAGTCTGTT |
| | | GCCGTCCTGGAAATGTACAACCTGTCCGTGGAGGACTCC |
| | | GGTGATTACACTTGCGAGGCGCACAATGCTGCTGGCAGC |
| | | GCCAGCTCTTCTACCAGCCTGAAAGTTAAAGAACCGCCG |
| | | GTTTTCCGTAAAAAGCCGCACCCGGTTGAAACTCTGAAA |
| | | GGTGCCGACGTGCATCTGGAATGTGAACTGCAGGGTACG |
| | | CCACCGTTTCAGGTTTCCTGGCACAAAGACAAACGTGAA |
| | | CTGCGCTCTGGCAAGAAATACAAAATTATGTCTGAAAAC |
| | | TTCCTGACGTCCATCCACATCCTGAACGTCGACTCCGCGG |
| | | ATATCGGCGAATATCAATGCAAAGCGTCTAACGATGTTG |
| | | GCTCTGACACCTGCGTGGGTTCTATCACCCTGAAAGCCCC |
| | | GCCTCGTTTCGTGAAGAAACTGTCTGATATTAGCACGGTC |
| | | GTTGGCGAAGAGGTTCAGCTGCAGGCTACTATCGAAGGT |
| | | GCGGAACCGATCTCCGTTGCATGGTTTAAAGATAAAGGT |
| | | GAGATCGTACGTGAATCCGATAACATCTGGATTTCCTACT |
| | | CTGAAAACATCGCGACTCTGCAATTCTCTCGTGCAGAACC |
| | | GGCGAACGCTGGCAAATACACCTGTCAGATCAAAAACGA |
| | | AGCGGGTACTCAGGAATGTTTTGCTACCCTGTCTGTTCTG |
| | | GAATCCGGA |
| gp41-1 | N-terminal | ATGGCTAAGACTAAAATGCTGAAAAAAATTCTGAAGATC |
| Int^{C} | split-intein | GAAGAACTGGACGAACGTGAACTGATTGACATCGAAGTG |
| (SEQ ID | | AGCGGTAACCATCTGTTCTACGCGAACGACATCCTGACCC |
| NO. 2) | | ACAACTCTTCTAGCGATGTTGGTACC |
| gp41-1 | C-terminal | ACCCGTTCTGGTTACTGTCTGGACCTGAAGACCCAGGTGC |
| Int^{N} | split-intein | AGACTCCACAGGGTATGAAAGAAATCTCTAACATCCAGG |
| (SEQ ID | | TTGGTGATCTGGTTCTGTCCAACACTGGCTATAACGAAGT |
| NO. 3) | | TCTGAACGTTTTCCCGAAATCCAAGAAAAAATCTTACAAA |
| | | ATCACCCTGGAAGATGGCAAAGAAATCATTTGCTCCGAA |
| | | GAACACCTGTTCCCGACCCAGACGGGCGAAATGAACATT |
| | | TCCGGCGGTCTGAAGGAAGGTATGTGCCTGTACGTGAAA |
| | | GAATAA |

TABLE 5
Plasmids.

| Plasmid Name | ORI | Promoter | Resistance | Description | Source |
|---|---|---|---|---|---|
| pE8k | pColEI | P_{BAD} | Kan^{R} | BglBricks | Previous |
| | | | | vector | study |
| pB6c | pBBR1 | P_{LacO1} | Cm^{R} | BglBricks | Previous |
| | | | | vector | study |
| p-1-4XT-1_{B} | pColEI | P_{BAD} | Kan^{R} | Plasmid pE8k | Present |
| | | | | containing | disclosure |
| | | | | sequence 4XT | |
| | | | | flanked by | |
| | | | | complimentary | |
| | | | | split-inteins | |
| | | | | gp41-1 Int^{N} and | |
| | | | | Int^{C} | |
| p-1-4XT-1_{L} | pBBR1 | P_{LacO1} | Cm^{R} | Plasmid pB6c | Present |
| | | | | containing | disclosure |
| | | | | sequence 4XT | |
| | | | | flanked by | |
| | | | | complimentary | |
| | | | | split-inteins | |
| | | | | gp41-1 Int^{N} and | |
| | | | | Int^{C} | |
| p-4XT | pBBR1 | P_{LacO1} | Cm^{R} | Plasmid pB6c | Present |
| | | | | containing | disclosure |
| | | | | sequence 4XT | |
| | | | | without any | |
| | | | | split-inteins | |
| p-8XT | pBBR1 | P_{LacO1} | Cm^{R} | Plasmid pB6c | Present |
| | | | | containing | disclosure |
| | | | | sequence 8XT | |
| | | | | without any | |
| | | | | split-inteins | |
| p-12XT | pBBR1 | P_{LacO1} | Cm^{R} | Plasmid pB6c | Present |
| | | | | containing | disclosure |
| | | | | sequence 12XT | |
| | | | | without any | |
| | | | | split-inteins | |

Bioproduction in shake flask cultures. Overnight seed cultures of 50 mL TB medium were inoculated with single colonies carrying the desired construct. These seed cultures were then used to inoculate cultures of 500 mL TB in 2 L Erlenmeyer flasks at an initial OD₆₀₀ of 0.08. Cultures were placed on reciprocal shakers at 350 rpm at 37° C. until OD₆₀₀ reached 3.0, at which point the corresponding inducer was added (0.2% arabinose or 1 mM IPTG for p-1-4XT-1 and p-4XT, respectively). Cultures were then continued at 37° C. for 20 h.

Bioproduction in fed-batch bioreactors. Both titin oligomers and polymer were ultimately produced in 2 L fed-batch bioreactors (Bioflo120, Eppendorf). Transformants were cultured overnight in 50 mL TB medium at 37° C. on an orbital shaker. The overnight cultures were then used to inoculate an autoclaved 2 L Bioflo120 heat-blanketed bioreactor containing 1.5 L M9 glucose medium with tryptone supplement (described herein above). Antifoam 204 was added as needed to minimize foaming (approximately 0.01% v/v). Agitation and air flow were regulated to maintain approximately 70% dissolved oxygen (DO). After consumption of the initial 0.5% w/v glucose (as judged by ΔDO), a sterile substrate feed (20% w/v glucose, 48 g/L tryptone, and 10 g/L MgSO₄·7H₂O) was initiated to maintain a linear growth rate. Reactors were induced at OD₆₀₀=70 by addition of 1 mM IPTG, and the incubation temperature was reduced to 30° C. Cultures were collected six hours after induction.

Protein purification. Polymer titin was purified by re-suspending cell pellets in urea lysis buffer (8 M urea, 300 mM NaCl, 10 mM imidazole, 20 mM KH₂PO₄. pH 7.4) at a ratio of 100 mL buffer to 50 g wet cell pellet weight. The solution was sonicated on ice using a QSonica Q700 sonicator (Qsonica) for 5 min (5 s on, 10 s off). Sonicated lysate was then pelleted by centrifugation at 25,000×g for 30 min. Cleared supernatant was sonicated an additional 5 min and then filtered through a 0.45 μm PES filter and applied to a series of six HisTrap HP 5 mL columns on an ÄKTA Pure Chromatography System (GE Healthcare Life Sciences) at a flow rate of 2 mL/min. Loaded columns were washed with two column volumes of lysis buffer, then washed by two column volumes of lysis buffer with 50 mM imidazole, and finally eluted by lysis buffer with 300 mM total imidazole.

The titin oligomers were purified by dissolving cell pellets in aqueous lysis buffer (50 mM Tris, 50 mM NaCl, 1 mM PMSF, and 300 μg/mL lysozyme). After stirring for 30 min at 4° C., 5 mM MgCl₂ and 5 μg/mL DNaseI were added, and the mixture was sonicated with stirring on ice for 10 min (5 s on, 10 s off). After sonication, NaCl and imidazole were added to final concentrations of 300 mM and 10 mM, respectively. The mixture was centrifuged at 25,000×g for 30 min at 4° C., followed by 75,000×g for 30 min at 4° C. Cleared supernatant was then filtered and applied to a series of six HisTrap HP 5 mL columns at 2 mL/min. Loaded columns were washed with 2 column volumes of wash buffer (50 mM Tris, 300 mM NaCl, 10 mM imidazole), then washed with 2 column volumes wash buffer with 50 mM imidazole, and finally eluted with wash buffer with 300 mM imidazole. After purification by affinity chromatography, samples were fully dialyzed to 5 mM ammonium bicarbonate at 4° C. using 10 kDa MWCO snakeskin dialysis tubing (Thermo Fisher Scientific).

SDS-PAGE. All SDS-PAGE gels were 1 mm thick, discontinuous with 3% stacking gel, and hand cast at the indicated percentages. Samples were prepared at 1 mg/mL total protein in Laemmli sample buffer (2% SDS, 10% glycerol, 60 mM Tris pH 6.8, 0.01% bromophenol blue, and 100 μM DTT). Gels were run on Mini-PROTEAN Tetra Cells (Bio-Rad) in 1× Tris-glycine SDS buffer (25 mM Tris base, 250 mM glycine, and 0.1% w/v SDS), until just before the dye front exited the gel. For MW estimation, Precision Plus Dual Color Prestained Standards (Bio-Rad) and HiMark Pre-stained Standards (ThermoFisher) were employed. Gels were stained in Coomassie Blue solution (50% v/v methanol, 10% v/v acetic acid, and 1 g/L Coomassie Brilliant Blue) for a minimum of one hour at room temperature with gentle agitation and destained in Coomassie Blue destain buffer (40% v/v methanol and 10% v/v acetic acid) for a minimum of one hour. Gels were imaged on an Azure c600 Imager (Azure Biosystems).

Analytical SEC. Protein was concentrated to approximately 10 mg/ml based on absorbance at 280 nm. A Superose 6 Increase 10/300 column (GE Healthcare) was equilibrated with elution buffer (10 mM potassium phosphate, 150 mM NaCl, and pH 7.4), after which 100 μL of sample were injected onto the column at 0.5 μL/min. The column was then eluted with 1 column volume of elution buffer, and the absorbance of the eluent was measured at 280 nm. Following the same procedure, 100 μL of protein standard mix (MilliporeSigma) and blue dextran (2000 kDa, MilliporeSigma) were separately passed through the column. A calibration curve was prepared by plotting the known MW of the standards against their retention volume (Vᵣ) divided by the void volume (V₀, blue dextran retention volume). An exponential curve was fit to the calibration data and used to calculate the MW of the titin polymer and oligomer based on their measured retention volumes. Polymer number-average MW (Mₙ) was calculated as Mₙ=ΣᵢMᵢNᵢ/ΣᵢNᵢ, where (Mᵢ) was taken as the calculated MW at a given data point on the polymer chromatogram (including only data from 1 kDa to 5 MDa) and Nᵢ was taken as the measured absorbance at the corresponding data point. Weight-average MW (M_{w}) was calculated as

$${M}_{w} = \frac{{Σ}_{i} {N}_{i} {M}_{i}^{2}}{{Σ}_{i} {N}_{i} {M}_{i}} .$$

Circular dichroism. CD spectra were acquired using a JASCO J-810 CD spectrometer equipped with a Lauda RM 6 refrigerated circulator and a JASCO PTC-423S peltier. Samples were diluted in 5 mM NaHCO₃ and loaded into a 1 mm quartz cuvette (Hellma, Germany). The CD spectra were obtained at 20° C., scanning in 1 nm steps from 190 to 260 nm with a 1 nm bandwidth, scanning speed of 100 nm/min, and 2 s response time. Multiple spectra were acquired, each the average of triplicate scans.

Scanning transmission electron microscopy. Samples (10 μL) were pipetted onto pure carbon, 400-mesh copper grids (Ted Pella, Inc.) that had been ozone-treated for 15 min using a Novascan PSD Series UV Ozone System (Novascan). After incubating for 5 min, grids were washed with 3 drops of ultrapure water and a 10 μl drop of 0.75% uranyl formate. Grids were then stained by adding a 10 μl drop of 0.75% uranyl formate and incubating for 3 min. Filter paper was used to wick liquids away between each step, and the grids were allowed to air-dry before imaging. Samples were imaged on a scanning transmission electron microscope (STEM, JEM-2100F, JEOL, Japan) set at 200 KV. STEM images were simultaneously recorded from both a bright-field (BF) and a high-angle annular dark-field (HAADF) detector. Using ImageJ software (v. 1.52a), fibril cross-sectional diameters were measured approximately every 10 nm along the fibril axis, avoiding regions with ambiguous stain boundaries. A total of 376 diameter measurements were made.

Fiber spinning. Fiber spinning was performed by first dissolving lyophilized titin powder in hexafluorisopropanol (HFIP) to 20% w/v. This protein dope was loaded to a 100 μL Hamilton gastight syringe (Hamilton Robotics) fitted with a 23s gauge (116 μm inner diameter and 4.34 cm length) needle. The syringe was fitted to a Harvard Apparatus Pump 11 Elite syringe pump (Harvard Apparatus), and the dope was extruded into a water bath at 5 μL/min. Extruded fibers were then carefully extended by hand in water at approximately 1 cm/s to 5× their original length. Extended fibers were removed from the bath and held under tension until visibly dry.

Light microscopy. Fiber diameters were measured using images acquired with a Zeiss Axio Observer ZI Inverted Microscope equipped with a phase contrast 20× objective lens and the Axiovision LE software (Zeiss).

Scanning electron microscopy. Following tensile tests, titin fibers were mounted onto a sample holder using double sided conductive tape (Electron Microscopy Sciences). The sample holder was sputter coated with a 10 nm gold layer using a Leica EM ACE600 high vacuum sputter coater (Leica Microsystems). Fibers were imaged using a Nova NanoSEM 230 Field Emission Scanning Electron Microscope (Field Electron and Ion Company, FEI) at an accelerating voltage of 7-10 kV.

Fourier transform infrared spectroscopy. For secondary structure determination, FT-IR spectra were acquired with a Thermo Nicolet 470 FT-IR spectrometer (Thermo Fisher Scientific) fitted with a Smart Performer ATR accessory with Ge crystal. Spectra were acquired from 1415-1780 cm⁻¹ at 2 cm⁻¹ resolution. A total of 254 scans were accumulated for each sample. All recorded spectra were analyzed using Fityk 0.9.8. Baselines were subtracted from all spectra using the built-in Fityk convex hull algorithm. The amide I band (1600-1700 cm⁻¹) was deconvolved into a set of eleven Lorentzian peaks centered at 1610, 1618.5, 1624.5, 1632.5, 1642, 1651, 1659, 1666.5, 1678, 1690.5, and 1700 cm⁻¹, corresponding to amide I shifts characteristic of β-sheet, random coil, α-helix, or β-turn structures, as described previously. Peak areas were integrated, and component percentages were calculated as the component peak area over the sum of all peak areas. Percentages were averaged from measurements of three fibers for each condition (as-spun and post-spin drawn). To directly compare spectra, each individual spectrum was normalized to the highest measured absorbance. Normalized spectra were averaged (three spectra for each condition) and overlaid.

Polarized Raman spectromicroscopy. The method reported here is adapted from several previous studies of molecular alignment in spider silk fibers. Titin fibers were carefully fixed to glass microscope slides with microscale markings to ensure that spectra were acquired at the same location before and after stage rotation. Raman spectra were acquired with a Renishaw RM1000 InVia Confocal Raman Spectrometer (Renishaw) coupled to a Leica DM LM microscope with rotating stage (Leica Microsystems). Fibers were initially oriented along the x-axis (parallel to the laser polarization). Fibers were irradiated at a fixed point with the 514 nm line of an argon laser with polarization fixed along the x-axis and focused through a 50× objective (NA=0.75). Spectra were recorded from 1100-1800 cm⁻¹ with an 1800 lines/mm grating. For each acquisition, a total of 10 spectra were accumulated, each for 10 s. The stage was then rotated to orient fibers along the y-axis with the same laser polarization, and spectra were acquired a second time at the same fixed point. No signs of thermal degradation were apparent, either visually or within recorded spectra. All recorded spectra were analyzed using Fityk 0.9.8. Baselines were subtracted from all spectra using the built-in Fityk automatic convex hull algorithm. For intensity ratio calculations, all spectra were normalized to the intensity of the 1450 cm⁻¹ peak, which arises from CH2 bending and is insensitive to protein conformation. For each fiber, the normalized intensity of the peak at 1670 cm⁻¹ when oriented along the Y-axis was divided by the normalized intensity of the peak when oriented along the X-axis to give the intensity ratio

$$I = \frac{Y}{X} .$$

This procedure was performed on a total of three separate fibers for each condition, and calculated intensity ratios were averaged. Spectra were also averaged and are presented with standard deviations for each point along the spectra.

Wide-angle X-ray diffraction data collection. Synchrotron-based wide-angle X-ray diffraction (WAXD) analysis was performed on the BioCars 14-BM-C beamline at the Advanced Photon Source at Argonne National Laboratory, Argonne, IL. The wavelength of the X-ray beam was 0.886 Å, with fixed energy of 14 keV, and the beam size was 130×340 μm² (horizontal×vertical). 2D diffraction images were recorded using a Pilatus3 S 2M Detector and the samples-to-detector distance was 200 mm. CeO₂ powder was used for the instrument calibration. For X-ray fiber diffraction measurements, the air background was measured first with no sample mounted on the sample stage. Then, bundles of 25 fibers, 1 mm in length and approximately 10 μm in diameter, were mounted across the opening of a rectangular paper frame. The assembly was loaded onto the sample stage with the fiber axis perpendicular to the X-ray beam, and the exposure time was 60 s to obtain a 2D diffraction image. The obtained diffraction intensities were subtracted by the air background intensity. Multiple images (≥3) were taken to improve the signal/noise ratio.

Wide-angle X-ray diffraction data analysis. To analyze the WAXD results, radial and azimuthal 1D profiles were sequentially obtained from the deconvolution of 2D diffraction images using the FIT2D software. The deconvolution and fitting of 1D profiles were performed with the peak analyzer tool in the OriginPro 2016 software (OriginLab, Northampton, MA). The data were fitted with Gaussian functions using nonlinear least squares fitting. 1D radial profiles of intensity versus scattering vector q (Å⁻¹, radius within the 2D diffraction image) were obtained by integrating azimuthally over a sector typically 20-30 degrees wide along either the equator or meridian.

The WAXD analysis assumed an orthorhombic unit cell commonly applied to β-sheet crystallites in semi-crystalline fibers. In particular, the 1D radial intensity profile along the equator includes two main equatorial Bragg reflections. Here the innermost equatorial peak is indexed as (200), corresponding to inter-sheet d-spacing along the unit cell a-axis and the outermost equatorial peak is indexed as (120), corresponding to inter-chain d-spacing along the unit cell b-axis (FIGS. 10A and 10B). After deconvolution of the 1D profile, the peak center (PC) was obtained, full width at half maximum (FWHM), and relative intensity (I) of crystalline peaks vs. amorphous components (FIG. 4H, FIG. 4I, Table 2). The degree of crystallinity was estimated by dividing the intensities of crystalline peaks by the sum of intensities from crystalline peaks and amorphous components (Table 2 and >Table 3)

$$( Crystallinity \% = \frac{{I}_{Equatorial ( 200 )} + {I}_{Equatorial ( 120 )}}{{I}_{Equatorial ( 200 )} + {I}_{Equatorial ( 120 )} + {I}_{EquatorialAmorphous 1} + {I}_{EquatorialAmorphous 2 )}} ) .$$

The center positions of the (200) and (120) crystalline peaks indicates a-axis inter-sheet d-spacing of 1.08 nm and b-axis inter-chain d-spacing of 0.46 nm, respectively (FIGS. 3A-C, Table 2, and Table 3). From the center position and FWHM of the (200) and (120) peaks, the Scherrer equation was used to determine the average crystallite size of 1.08 nm along the inter-sheet a-axis and 2.91 nm along the inter-chain b-axis, respectively (FIGS. 10A and 10B, Table 7, and Table 8). The Scherrer equation is expressed by

$$D = \frac{K λ}{βcos θ} .$$

Where D is the mean size of the crystallite domains, K is a dimensionless shape factor (with a typical value of 0.9), λ is X-ray wavelength (0.886 nm), β is FWHM value in radians (conversion of the FWHM to radians is using

$$β = 2 arc sin ( \frac{λ \times FWHM}{4 π} ) ,$$

λ is X-ray wavelength), and θ (*) is the Bragg angle). Conversion of the PC disclosed herein to Bragg angle is using

$$θ = arc sin ( \frac{λ \times PC}{4 π} ) \times \frac{360}{2 π} ,$$

λ is X-ray wavelength. Because the calculated average crystallite size along the inter-sheet a-axis is the same as the d-spacing, this indicates that β-crystals contain two β-sheets.

TABLE 6
Primer sequences.

| Primer Name | Description | Sequence |
|---|---|---|
| prF1 (SEQ ID NO. | Primer for adding | tttttccggaGCTAGCGGTACCCACCACCATCACC |
| 4) | Kpn2I restriction | |
| | site to 5′ | |
| | end of 4XT | |
| | sequence. Used in | |
| | constructing p- | |
| | 8XT and p-12XT. | |
| prF2 (SEQ ID NO. | Primer for adding | tttttccggaGCTAGCGGTACCCACCACCATCACC |
| 5) | NheI restriction site | |
| | to 5′ | |
| | end of 4XT | |
| | sequence. Used in | |
| | constructing p- | |
| | 12XT. | |
| prR1 (SEQ ID NO. | Primer for | tttttccggaGGATCCTTATTCCAGAACAGACAGGGTAG |
| 6) | maintaining stop | |
| | codon and BamHI | |
| | restriction site on 3′ | |
| | end of 4XT | |
| | sequence. | |
| | Used in | |
| | constructing p-8XT | |
| | and p-12XT. | |
| prR2 (SEQ ID NO. | Primer for adding | tttttccggaACTAGTTTCCAGAACAGACAGGGTAGC |
| 7) | Spel restriction site | |
| | to 3′ | |
| | end of 4XT | |
| | sequence. Used in | |
| | constructing p- | |
| | 12XT. | |

TABLE 7
Strains.

| Strain Name | Description | Source |
|---|---|---|
| NEB10β | F′ proA + B + lacIq Δ(lacZ)M15 zzf::Tn10 | Previous study |
| | (TetR) Δ(ara-leu) 7697 araD139 fhuA | |
| | ΔlacX74 | |
| | galK16 galE15 e14- Φ80dlacZΔM15 recA1 | |
| | relA1 endA1 nupG rpsL (StrR) rph spoT1 | |
| | Δ(mrr-hsdRMS-mcrBC) | |
| s-1-4XT-1_{B} | NEB10β containing plasmid p-1-4XT-1_{B} | Present disclosure |
| s-1-4XT-1_{L} | NEB10β containing plasmid p-1-4XT-1_{L} | Present disclosure |
| s-4XT | NEB10β containing plasmid p-4XT | Present disclosure |
| s-8XT | NEB10β containing plasmid p-8XT | Present disclosure |
| s-12XT | NEB10β containing plasmid p-12XT | Present disclosure |

TABLE 8
Mechanical properties for different microbially produced (rows 2-
10), natural (rows 11-19), and manmade (rows 20-26) materials.

| | | Tensile Strength | Toughness | Damping |
|---|---|---|---|---|
| Row | Material | (MPa) | (MJ/m³ ) | capacity (%) |
| 1 | Microbially Produced Titin | 378 | 130 | 81 |
| | (present disclosure) | | | |
| 2 | Bacterial cellulose (BC) | 0.12-0.68 | 0.016-0.080 | — |
| 3 | GB1-resilin muscle mimic | 0.026-0.057 | 0.004-0.014 | 13-18 |
| 4 | Poly(3-hydroxybutyrate-co- | 38.8-44.6 | 1.2* | — |
| | 3-hydroxyvalerate) (PHBV) | | | |
| 5 | Poly(3-hydroxybutyrate-co- | 36.2-39.2 | 1.7-8.9* | — |
| | 3-hydroxyvalerate)-Poly(ε- | | | |
| | caprolactone) (PHBV-PCL) | | | |
| 6 | Polyhydroxybutyrate (PHB) | 14.3-32.1 | 0.244-2.00 | — |
| 7 | Poly(3-hydroxybutyrate-co- | 17-65 | — | 40.6 |
| | 4-hydroxyvalerate) (P34HB) | | | |
| 8 | Polyhydroxybutyrate- | 24.5-27.5 | 0.21-0.99* | — |
| | polypropylene (PHB-PP) | | | |
| 9 | Poly(lactic acid) (PLA) | 21-60 | 2 | — |
| 10 | ε-Poly-L-lysine- | 3.1-27.9 | 0.4-8.4 | — |
| | Lignosulfonate (ε-PL- | | | |
| | Lignosulfonate) | | | |
| 11 | Bone (mineralized collagen) | 110-875 | 4 | — |
| 12 | Cotton | 205-778 | 4.27-30.1 | 45-72 |
| 13 | Elastin | 0.306-1.38 | 2 | 24 |
| 14 | Resilin | 3.03-4.25 | 4 | 3 |
| 15 | Silkworm silk | 540-592 | 47-64 | 48-65 |
| 16 | Spider silk (dragline) | 544.59-1469.34 | 47.85-230.02 | 41-75 |
| 17 | Tendon (collagen) | 45.9-182 | 1-1.78 | 3-20 |
| 18 | Viscose/rayon | 304-662 | 32.0-40.1 | 65-80 |
| 19 | Wool | 37.4-206 | 24.3-47.6 | 31-65 |
| 20 | Carbon fiber (CF) | 4000 | 25 | — |
| 21 | Kevlar | 3600 | 60 | — |
| 22 | Nylon | 750-950 | 63.4-107 | 5-40 |
| 23 | Polypropylene (PP) | 20.9-37.1 | 170 | 8-35 |
| 24 | Polyurethane (PU) | 18.8 | 79.5 | — |
| 25 | Steel | 1650 | 6 | — |
| 26 | Synthetic rubber | 50 | 100 | 76.2 |

*For materials that did not report toughness data, a toughness “upper limit” was calculated using the reported mechanical properties.

To estimate the degree of orientation of the crystallites along the fiber axis, two azimuthal 1D profiles were obtained from the 2D diffraction image (FIG. 4J, FIG. 4K). FIG. 4J shows the plot of the diffraction intensity integration as a function of azimuthal angle within the radial range of the equatorial (120) peak, and FIG. 4K is the corresponding plot within the radial range of the equatorial (200) peak. After deconvolution of the ID profiles, the calculated FWHMs of the crystalline peaks and amorphous components to the Herman's orientation function ƒ_{crystal}=(3<cos²φ>−1)/2 were applied (see Table 2 and Table 3). Here φ is the angle between the c-axis and the fiber axis and <cos²φ> is obtained based on the equation <cos²φ>=1-0.8<sin²(0.4×FWMH₍₂₀₀₎>−1.2<sin² (0.4×FWMH₍₁₂₀₎)>. The parameter f_{crystal} is 0 for no preferred orientation and 1 if all crystallites are perfectly aligned.

Fiber mechanical testing and cyclic loading measurements. Segments of post-drawn fibers (20 mm) were carefully laid exactly vertical across a 5 mm (vertical)×15 mm (horizontal) opening cut into a 20 mm×20 mm piece of cardstock and fixed with adhesive tape at both ends of the opening. Diameters of mounted fibers were then measured by light microscopy, averaging measurements at three points along the fiber axis. Mechanical properties were measured by axial pull tests on an MTS Criterion Model 41 universal test frame fitted with a 1 N load cell (MTS Systems Corporation). Cardstock holders were mounted between two opposing spring-loaded grips, and the supporting edges were carefully cut. Pull tests were conducted at a relative humidity of 45% and temperature of 22° C., with a constant crosshead speed of 10 mm/min. Stress-strain curves were recorded by the MTS TW Elite test suite at a sampling rate of 50 Hz. Fiber breaks were recorded when a 90% drop from peak stress was detected. All mechanical properties were automatically calculated by the MTS TW Elite test suite. Ultimate tensile strength was calculated as the maximum measured load over the initial fiber cross-sectional area (A=πr²), as determined from measured initial diameters. Modulus was calculated as the slope of a linear least squares fit to the stress/strain data of the initial elastic region. Toughness was calculated as the area under the total stress/strain curve divided by the initial fiber volume (V=πr²h), as calculated from measured initial fiber diameters and set initial gage length of 5 mm. For each protein, a total of 14 fibers were measured in this manner.

Fibers were prepared and mounted as described above. Fibers were pulled to 30% elongation and returned to 0% elongation at a rate of 10 mm/min, and load was measured with a 1 N load cell. Damping capacity was calculated as the ratio of the energy between the loading and unloading curves over the energy under the loading curve. Damping energy was calculated as the energy difference between the loading and unloading curves divided by the initial fiber volume, as calculated from measured initial fiber diameters and set initial gauge length of 5 mm.

Molecular dynamics simulation. The representative simulation volume of the fiber was constructed within the Large-scale Atomic/Molecular Massively Parallel Simulator (LAMMPS) with periodic boundary conditions to simulate bulk behavior. VMD (Visual Molecular Dynamics) was used for the visualization of these systems. Within the periodic box, an initial molecular structure was assembled based on structural analyses of the real fiber. In detail, the all-atomistic structure of titin oligomers (167-170, 4Ig) was first extracted from protein structure 3B43 in Protein Data Bank. The psf file was generated using the “Automatic PSF Builder” toolkit in VMD. The four linked Ig domains form an oriented fibril. The 4Ig fibrils were aligned along the model y-axis and arranged 2×2 stacks of fibrils, with each 4Ig fibril positioned antiparallel to its flanking 41 g fibril and offset by two Ig domains (FIGS. 14A, 14B, 14C, and 14D). Then the pdb/psf files were converted into a LAAMPS input file. Sodium ions were then added into the simulation box to neutralize the system. The CHARMM36 force field was used.

After minimizing the system energy for 10,000 time steps in LAMMPS, the system was first equilibrated in the NPT ensemble for 1 ns. Then at room temperature, the titin fiber was compressed in the x and z directions (perpendicular to the fiber axis) from 1 bar to 4,000 bars and relaxed back to 1 bar in 2 ns, followed by equilibration of 5 ns. The temperature and pressure damping parameters were chosen as 0.1 ps and 1 ps, respectively. The applied high pressure is to ensure that side-surfaces of the fibrils have close contact with each other so that adjacent Ig-like domains are paired to pack into the same crystalline domain as indicated by WAXD results (FIGS. 10A and 10B). The β-sheet content in the final model structure was measured to be 22%, in agreement with FT-IR analysis.

To carry out uniaxial tensile tests on the titin fiber, a non-equilibrium MD (NEMD) simulation was conducted in NPₓP_{z}T ensemble, with x and z directions in the atmosphere pressure. Uniaxial strains were applied to the y direction (fiber axis) with a constant strain rate 1×10⁸/s. LAMMPS output the engineering strain and the stress, which consists of a kinetic energy term and the virial term (from interactions between atoms, such as pair, bond, angle, and dihedral contributions). All simulations were run three times.

To measure the intra- and inter-fibril bonded and non-bonded interaction energies, the static structures calculated from LAMMPS were input into the NAMD software. Every structure was equilibrated for 100 ps and run for another 100 ps for energy calculation. The ‘NAMD Energy’ toolkit was used in VMD to output every intra- and inter-fibril interaction term. The hydrogen bonds were calculated using the ‘Hydrogen Bonds’ toolkit in VMD, with only polar atoms (N, O, S) considered. The donor-acceptor distance and angle cutoff were 3.8 Å and 30 deg, respectively. The salt bridges were calculated using the ‘Salt Bridges’ toolkit in VMD, with O-N cutoff distance 3.5 Å. The atomic stress was output from the ‘stress/atom’ command in LAMMPS, which also consists of kinetic energy term and the virial term.

CD Spectral Analysis. CD spectra were analyzed with the BestSel Single Spectrum Analysis and Fold Recognition tool (http://bestsel.elte.hu/index.php, last accessed Aug. 9, 2020). In brief, this algorithm estimates the secondary structure content of the sample spectrum by fitting to spectra of proteins with known structures. All proteins in the database are indexed based on proportions of eight different secondary structure elements (regular alpha-helix, distorted alpha-helix, left-twisted antiparallel beta-strand, relaxed antiparallel beta-strand, parallel beta strand, turn, and others). Each structure is represented as a point in an eight-dimensional secondary structure space. The algorithm reports the predicted proportion of eight secondary structural elements for the sample spectrum and gives a list of the top ten structures closest to the predicted sample structure in this eight-dimensional space.

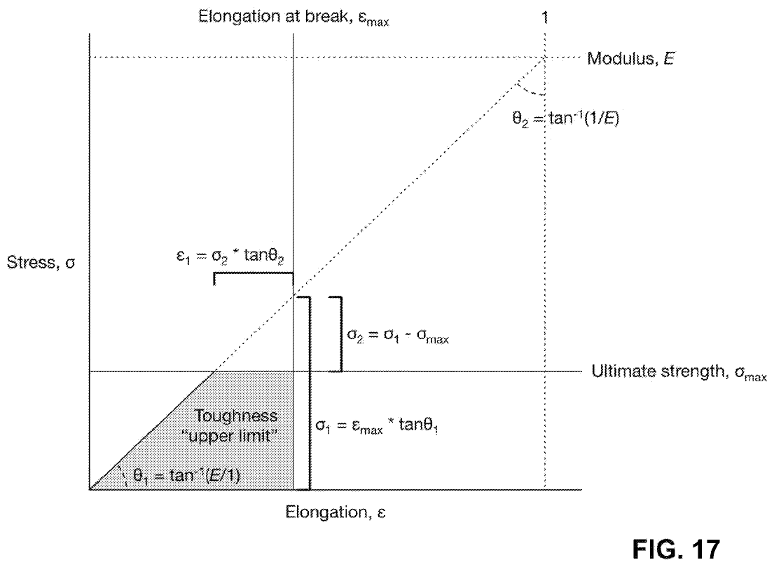

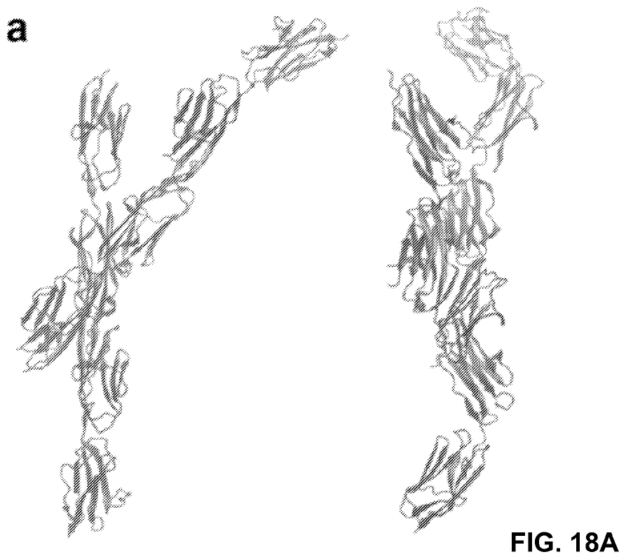

Toughness Upper-Limit Calculation. For materials that did not have a reported toughness, a toughness “upper limit” was calculated using the reported values for tensile strength, Young's modulus, and elongation at break. Because ultimate tensile strength defines the maximum measured stress, elongation at break defines the maximum measured strain, and Young's modulus defines the steepest measured slope of the curve (during the elastic regime), the following equations derived from FIG. 17 are used to calculate the maximum possible toughness value for any set of those three mechanical properties for a given material:

$${θ}_{1} = {tan}^{- 1} ( E / 1 ) \\ {θ}_{2} = {tan}^{- 1} ( 1 / E ) \\ {σ}_{1} = {ε}_{max} * tan {θ}_{1} \\ {σ}_{2} = {σ}_{1} - {σ}_{max} \\ {ε}_{1} = {σ}_{2} * tan {θ}_{2} \\ Toughness upper limit = ( {ε}_{max} * {σ}_{1} ) / 2 - ( {ε}_{1} * {σ}_{2} ) / 2. \qquad ( see FIG . 17 )$$

Macro-Scale Model. To construct a representative model of the macro-scale titin fiber, it was first assumed that the titin domains do not unfold appreciably during the fiber spinning process, as proteins are known to remain folded even under high shear flow. Protein docking GRAMM-X web server was then employed in attempt to establish an initial assembly of two titin chains (FIG. 18A). However, the energy minimized structures predicted using protein docking did not yield conformations with aligned chains as seen in the experimental data of the fibers of the present disclosure. This is likely because the docking results obtain a local minimum on the basis of equilibrium conditions and cannot take into account the surrounding protein chains and mechanical microenvironment present during spinning, which can induce greater packing and orientation. Specifically, high shear flow can significantly impact the supramolecular assembly of proteins. For example, amyloid proteins can transfer from spherical aggregates in low shear flow (\dot{γ}˜40/s) to thick fibers in high shear flow (\dot{γ}˜400/s). The shear flow is calculated as \dot{γ}˜500/s, assuming therefore that the titin chains will be well-aligned along the fiber axis. This assumption is supported by experimental results from Raman and X-ray diffraction analysis. This, along with the observed large initial modulus of real titin fibers, indicates that the bending motion of individual titin chains is likely hindered due to lateral packing into bundles through close interactions between chain surfaces. Hence, a lateral pressure was applied to compress the initially loosely assembled titin fiber in the simulation. Additionally, the importance of the alignment of single titin chains in the simulated titin fiber was also noted. The staggered assembly pattern allows the shear force transmission between adjacent chains, which ensures the high stiffness and toughness of the bulk fiber. In comparison, if the chains stack face-to-face or in any other orientation, then the stress cannot transmit across the fiber, resulting in a very weak fiber (FIG. 18C). Therefore, a representative model was chose with two Ig domains stacked into a well packed configuration. The inter-fibril interaction energy calculated from this configuration (−1415 kcal/mol, FIG. 18B) is more favorable than the structures obtained from GRAMM-X (−1009 kcal/mol, FIG. 18A).

Between interfaces of equilibrated titin chains, there exist Vdw and electrostatic interactions, which change slightly during the fiber stretching. The solvent-accessible surface (SASA) was also calculated of a titin chain buried in surrounding titin chains using VMD. SASA of the single chain is SI, SASA of the single chain with surrounding chains is S2, and SASA of the complex without the single chain is S3. The single chain interaction area ratio is defined by (S3+S1-S2)/2S1, which is calculated as 74% for the disclosed titin. In contrast, if the chains are not compressed during equilibration, the interaction area ratio is calculated as 52%. Hence, the titin chains in the presently disclosed model interact extensively with other chains.

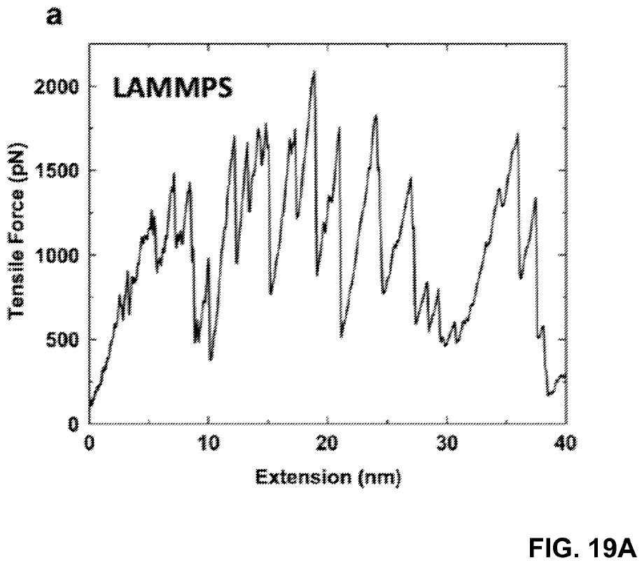

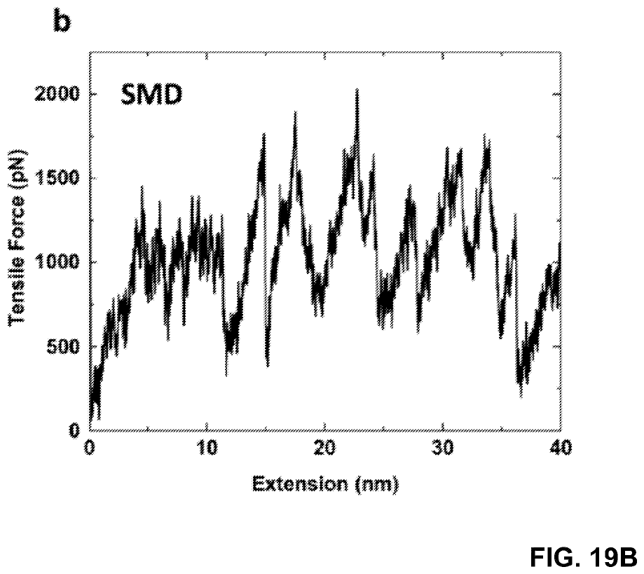

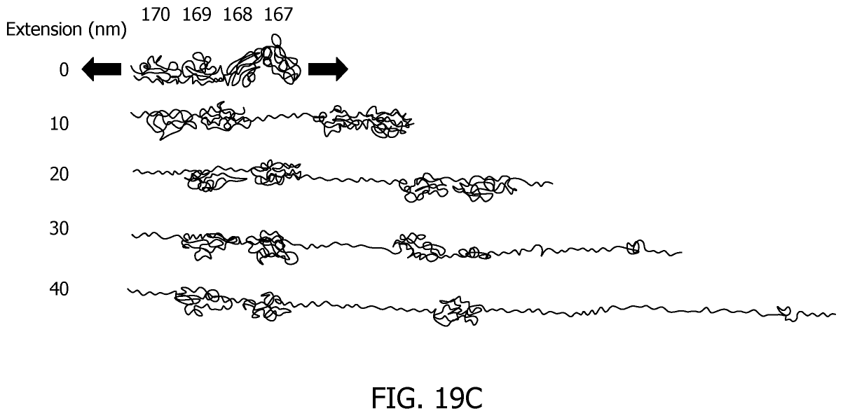

To compare the disclosed approach with constant velocity Steered MD (SMD), LAMMPS was first used with the ‘fix deform’ command to stretch a single titin chain (with the top and bottom of the chain linked across the periodic box boundary, such that this represents an infinite chain with repeats in image cells). The tensile strain rate was 1×108/s, which can be related to a pulling rate of 1.2 m/s that is comparable to previous studies with MD. FIG. 19A is the representative single titin fibril tensile force-displacement curve calculated using this approach. The overall shape of the curve matches the saw-tooth pattern from experiments and other SMD simulations for single chains, with gradual increases of force and sudden drops after peak force that indicate the cooperative unfolding of titin domains. The distance to fully unfold 167, the first Ig domain to unfold, is about 25 nm, which matches well with previous SMD simulations. The sequential unfolding of domains, shown in FIG. 19C, is also similar to previous SMD simulations. Furthermore, to validate this approach, SMD was also used in NAMD to pull a single titin chain with a pulling velocity of 1.2 m/s: the measured curve of tensile force (FIG. 19B) matches well the result measured with LAMMPS.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims. To facilitate the understanding of the embodiments described herein, a number of terms are defined below. The terms defined herein have meanings as commonly understood by a person of ordinary skill in the areas relevant to the present disclosure. Terms such as “a.” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

Any non-limiting examples are provided to further illustrate the present disclosure. It should be appreciated by those of skill in the art that the techniques disclosed in the examples represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

1. A microbially-synthesized titin protein comprising a plurality of folded immunoglobulin-like (Ig-like) domains.

2. The microbially-synthesized titin protein of claim 1, wherein the protein is an oligomer protein having at least about 4 Ig-like domains to at least about 20 Ig-like domains.

3. The microbially-synthesized titin protein of claim 2, wherein the oligomer protein has a molecular weight of at least about 40 kDa to at least about 150 kDa.

4. The microbially-synthesized titin protein of claim 1, wherein the protein is a polymer protein comprising more than about 20 Ig-like domains.

5. The microbially-synthesized titin protein of claim 4, wherein the polymer protein has a mass average molecular weight of at least 2 MDa, at least 3 MDa, at least 4 MDa, or at least 5 MDa.

6. A titin fiber comprising the microbially-synthesized titin protein of claim 1.

7. (canceled)

8. (canceled)

9. The titin fiber of claim 6 having a toughness of from about 15 MJ/m³ to about 75 MJ/m³, or from about 20 MJ/m³ to about 70 MJ/m³, or from about 115 MJ/m³ to about 150 MJ/m³, or from about 120 MJ/m³ to about 140 MJ/m³.

10. The titin fiber of claim 6 having an ultimate tensile strength of from 100 MPa to about 250 MPa, or from about 150 MPa to about 225 MPa, or from about 350 MPa to about 500 MPa.

11. (canceled)

12. (canceled)

13. (canceled)

14. (canceled)

15. A process for synthesizing fibers from at least one microbially-produced titin protein, the process comprising dissolving the at least one microbially-produced titin protein in a denaturing solvent to produce a protein dope solution, wherein the at least one microbially-produced titin protein has a mass average molecular weight of at least about 40 kDa to at least about 5 MDa.

16. The process of claim 15, wherein the at least one microbially-produced titin protein is selected from an oligomer protein having at least about 4 Ig-like domains to at least about 20 Ig-like domains having a molecular weight of at least about 40 kDa to at least about 150 kDa, a polymer protein comprising more than about 20 Ig-like domains having a mass average molecular weight of at least 2 MDa to about at least 5 MDa, and combinations thereof.

17. (canceled)

18. The process of claim 15, further comprising continuously spinning the microbially-synthesized titin polymer into fibers by extruding the polymer dope solution through a narrow-bore needle into a solvent.

19. The process of claim 18, wherein the solvent is an aqueous solution.

20. (canceled)

21. The process of claim 18, further comprising subjecting the fibers to a post-spin draw.

22. A method of synthesizing titin polymers, the method comprising:
flanking an oligomer protein with a split-intein pair, wherein the oligomer protein is a titin subunit protein sequence comprising spatially-separated termini, and wherein the split-intein pair comprises a C-terminal half and an N-terminal half;
overexpressing the flanked oligomer protein coding sequence in a protein expression system host cell culture to produce a chimeric Int^{C}-(oligomer protein)-Int^{N} protein; and
polymerizing the flanked oligomer protein in vivo by a plurality of successive rounds of split-intein-catalyzed intermolecular ligation to produce titin polymers having a mass average molecular weight of at least 2 MDa.

23. The method of claim 22, wherein the host cell is a protein-expressing microbial cell.

24. The method of claim 23, wherein the protein-expressing microbial cell comprises an Escherichia coli cell, a Bacillus subtilis cell, a Saccharomyces cerevisiae cell, a Pichia pastoris cell.

25. The method of claim 22, wherein the synthesized titin polymers comprise a plurality of folded Ig-like domains.

26. The method of claim 22, wherein the synthesized titin polymers have a mass average molecular weight of at least 2 MDa, at least 3 MDa, at least 4 MDa, or at least 5 MDa.

27. The method of claim 22, wherein the synthesized titin polymers have a toughness of from about 115 MJ/m³ to about 150 MJ/m³, or from about 120 MJ/m³ to about 140 MJ/m³.

28. The method of claim 22, wherein the synthesized titin polymers have an ultimate tensile strength of from 350 MPa to about 500 MPa.

29. (canceled)

30. (canceled)


SEQUENCE LISTING (7 sequences)

SEQ ID NO: 1 - DNA, 1152 bp, Artificial Sequence
  Codon optimized CDS for rabbit soleus titin domains I67-I70
ggtacccaccaccatcaccatcatccgccgttcttcgatctgaagccggtgtctgttgacctggctctgggtgaatctggcaccttcaaatgccacgtgaccggcaccgctccgatcaagattacgtgggcgaaagacaaccgcgagattcgtccaggcggcaactacaaaatgaccctggtggaaaacactgcgaccctgaccgtcctgaaagtgaccaaaggtgacgcgggccagtacacttgttacgcaagcaacgtggcgggcaaagattcttgctctgcacagctgggcgtgcaggaaccgcctcgcttcattaaaaagctggagccgtcccgcatcgtgaaacaggacgaacatacccgttatgaatgcaaaattggcggctctcctgaaatcaaagtactgtggtataaggacgaaaccgagatccaggagtcttctaagttccgtatgtctttcgtggagtctgttgccgtcctggaaatgtacaacctgtccgtggaggactccggtgattacacttgcgaggcgcacaatgctgctggcagcgccagctcttctaccagcctgaaagttaaagaaccgccggttttccgtaaaaagccgcacccggttgaaactctgaaaggtgccgacgtgcatctggaatgtgaactgcagggtacgccaccgtttcaggtttcctggcacaaagacaaacgtgaactgcgctctggcaagaaatacaaaattatgtctgaaaacttcctgacgtccatccacatcctgaacgtcgactccgcggatatcggcgaatatcaatgcaaagcgtctaacgatgttggctctgacacctgcgtgggttctatcaccctgaaagccccgcctcgtttcgtgaagaaactgtctgatattagcacggtcgttggcgaagaggttcagctgcaggctactatcgaaggtgcggaaccgatctccgttgcatggtttaaagataaaggtgagatcgtacgtgaatccgataacatctggatttcctactctgaaaacatcgcgactctgcaattctctcgtgcagaaccggcgaacgctggcaaatacacctgtcagatcaaaaacgaagcgggtactcaggaatgttttgctaccctgtctgttctggaatccgga

SEQ ID NO: 2 - DNA, 144 bp, Artificial Sequence
  N-terminal split-intein
atggctaagactaaaatgctgaaaaaaattctgaagatcgaagaactggacgaacgtgaactgattgacatcgaagtgagcggtaaccatctgttctacgcgaacgacatcctgacccacaactcttctagcgatgttggtacc

SEQ ID NO: 3 - DNA, 282 bp, Artificial Sequence
  C-terminal split-intein
acccgttctggttactgtctggacctgaagacccaggtgcagactccacagggtatgaaagaaatctctaacatccaggttggtgatctggttctgtccaacactggctataacgaagttctgaacgttttcccgaaatccaagaaaaaatcttacaaaatcaccctggaagatggcaaagaaatcatttgctccgaagaacacctgttcccgacccagacgggcgaaatgaacatttccggcggtctgaaggaaggtatgtgcctgtacgtgaaagaataa

SEQ ID NO: 4 - DNA, 35 bp, Artificial Sequence
  Primer for adding Kpn2I restriction site to 5′end of 4XT sequence. Used in constructing p-8XT and p-12XT.
tttttccggagctagcggtacccaccaccatcacc

SEQ ID NO: 5 - DNA, 35 bp, Artificial Sequence; identical to SEQ ID NO: 4
  Primer for adding NheI restriction site to 5′end of 4XT sequence. Used in constructing p-12XT.

SEQ ID NO: 6 - DNA, 39 bp, Artificial Sequence
  Primer for maintaining stop codon and BamHI restriction site on 3′ end of 4XT sequence. Used in constructing p-8XT and p-12XT.
tttttccggaggatccttattccagaacagacagggtag

SEQ ID NO: 7 - DNA, 37 bp, Artificial Sequence
  Primer for adding SpeI restriction site to 3′end of 4XT sequence. Used in constructing p-12XT.
tttttccggaactagtttccagaacagacagggtagc